(19) United States
(12) Patent Application Publication
(10) Pub. No.: US 2024/0167107 A1
(43) Pub. Date: May 23, 2024

(54) COMPOSITION AND METHOD FOR NUCLEIC ACID DETECTION

(71) Applicant: APTITUDE MEDICAL SYSTEMS, INC., Goleta, CA (US)

(72) Inventors: Jinpeng WANG, Goleta, CA (US); Hui KANG, Goleta, CA (US); Qin YANG, Goleta, CA (US); Brian Scott FERGUSON, Goleta, CA (US); Qiang GONG, Goleta, CA (US)

(73) Assignee: APTITUDE MEDICAL SYSTEMS, INC., Goleta, CA (US)

(21) Appl. No.: 18/546,358

(22) PCT Filed: Feb. 14, 2022
(86) PCT No.: PCT/US2022/016266

§ 371 Date: Aug. 14, 2023

Related U.S. Application Data
(60) Provisional application No. 63/149,984, filed on Feb. 16, 2021.

(51) Int. Cl. C12Q 1/70; C12Q 1/6816

(52) U.S. Cl. CPC C12Q 1/701; C12Q 1/6816

(57) ABSTRACT

The present application provides compositions and methods for detecting a target nucleic acid in a sample. The present application also provides systems, isolated oligonucleotides, and kits for detecting a target nucleic acid in a sample.

### BACKGROUND OF THE INVENTION

The outbreak of worldwide pandemic (e.g., those caused by pathogens, such as by virus/bacteria) has caused enormous social and economic issues. The potential large number of asymptomatic or pre-symptomatic carriers of the pathogens (e.g., a virus) who are unlikely to seek testing and may unknowingly spread the virus has become a major concern. Accordingly, there is an urgent need for decentralized population-scale testing that can be readily and reliably used at home or in public places to enable rapid identification of all infected individuals with or without symptoms, thereby minimizing the chances of new outbreaks.

Existing methods are not sufficient. Current virus testing systems and methods suffer from insufficient performance, low capacity, or in-accessibility.

For example, serology tests detect antibodies indicating infection that does not develop until at least a week after the onset of symptoms, thus, such tests are not useful for preventing the spread of the virus.

The viral antigen test detects the specific antigen on a virus and can be developed as a rapid test. However, it suffers from low sensitivity and high false negative rate for pre-symptomatic individuals who are already infectious, rendering it a less effective screening tool.

The molecular test detects viral RNA and can identify infected patients before they show any symptoms, it may be useful for population-scale testing to control the pandemic. Unfortunately, laboratory-based molecular tests require complex facilities, specialized costly instrumentation, well-trained technical staff, and is often time-consuming, thus incapable of meeting this population-scale testing demand. In addition to the capacity problem, lab-based molecular testing suffers from inaccessibility given logistical constraints created by centralized sample collection, transportation, and processing, hampering diagnosis at the point of need where isolation of infectious individuals can be immediately implemented to prevent the spread of the virus, such as in assisted living facilities, classrooms, airports, workplaces, shopping malls, other public places, or at home. In fact, all centralized testing approaches will inevitably cause delay from sample collection to reporting, increasing the critical latency to isolation of infectious individuals. While emerging point of care tests (POCT) such as the Abbott ID NOW™M can perform viral RNA tests in less than one hour in less-centralized medical facilities, they still must be operated by health-care professional (HCP) and require expensive instrumentation, limiting their accessibility and utility.

In addition, to develop a rapid, convenient, and safe test (especially when working with potentially highly infectious samples), it would be desired to avoid laborious sample processing. However, unprocessed samples are often very challenging to handle, for example, such samples may comprise a lot of interferences, rendering it difficult to amplify the target nucleic acids therein. Certain samples (e.g., samples comprising unstable nucleic acids, such as RNAs) need to be analyzed immediately after being obtained from a subject, which makes high throughput analysis less likely.

### SUMMARY OF THE INVENTION

The present application provides a composition and/or a system to address such unmet urgent needs. The composition and/or a system of the present application could rapidly detect viral nucleic acids (RNA and/or DNA) directly from unprocessed samples (e.g., saliva or swab samples obtained directly from a subject). The composition and/or a system of the present application could also be used for rapid (e.g., in less than 20 minutes), robust and unambiguous detection (e.g., diagnosis) of potential viral infections. In addition, the simple, sensitive, robust and rapid composition and/or a system of the present application would support use at home or in public settings by untrained users.

Further, the composition and/or a system of the present application is capable of one or more of the following:
(1) detecting a target nucleic acid (e.g., a virus nucleic acid, such as a virus RNA) present in a sample with 1000 copies or less (e.g., 100 copies or less);
(2) detecting all the published sequences of a target pathogen (e.g., a virus, such as SARS-COV-2), for example, the composition and/or a system of the present application is capable of detecting each of the 45,869 published sequences of SARS-COV-2;
(3) not cross-reacting with any agent having a sequence homology of about 80% or lower to the target nucleic acid; and
(4) stabilizing the sample and any target nucleic acid molecule within the sample.

In one aspect, the present application provides a composition for detecting a target nucleic acid in a sample, the composition comprising: a nuclease inhibitor and a decoy oligonucleotide.

In some embodiments, the decoy oligonucleotide is a non-target nucleic acid molecule capable of specifically binding to a nuclease.

In some embodiments, the decoy oligonucleotide is capable of specifically binding to an RNase.

In some embodiments, the decoy oligonucleotide comprises tRNAs.

In some embodiments, the decoy oligonucleotide does not comprise a modified nucleotide.

In some embodiments, the decoy oligonucleotide comprises natural tRNAs.

In some embodiments, the decoy oligonucleotide comprises natural tRNAs derived from a yeast.

In some embodiments, the decoy oligonucleotide comprises R8759 (Sigma Aldrich).

In some embodiments, the nuclease inhibitor comprises an RNase inhibitor.

In some embodiments, the RNase inhibitor comprises a protein-based RNase inhibitor and/or a chemical-based RNase inhibitor.

In some embodiments, the RNase inhibitor is capable of specifically inhibiting an activity of RNase A, RNase B and/or RNase C.

In some embodiments, the RNase inhibitor is capable of reducing one or more disulfide bonds in an RNase.

In some embodiments, the RNase inhibitor does not substantially inhibit an activity of a nucleic acid polymerase.

In some embodiments, the RNase inhibitor does not substantially inhibit an activity of a DNA polymerase, and/or an activity of a reverse transcriptase.

In some embodiments, the nuclease inhibitor comprises one or more of the following: oligovinylsulfonic acid (OVA), aurintricarboxylic acid (ATA), aflatoxin, 2-nitro-5-thiocyanobenzoic acid, iodoacetate, N-bromosuccinimide, p-chloromercuribenzoate, diethyl pyrocarbonate, ethanol, formamide, guanidinium thiocyanate (GdnSCN), dinitrofluorobenzene, decanavanate, polyvinylsufonic acid, hydrobenzoinphosphate, phenylphosphate, putrescine, haloacetate, dinitrofluorobenzene, phenylglyoxal, bromopyruvic, hydroxylamine-oxygen-cupric ion, a vanadyl complex, 8-amino-5-(4′-hydroxy-biphenyl-4-ylazo)-naphthalene-2-sulfonate, 6-hydroxy-5-(2-hydroxy-3,5-dinitro-phenylazo)-naphthalene-2-sulfonate, 3,3′-dimethylbiphenyl-4,4′-bis(2-amino-naphthylazo-6-sulfonate), 4,4′-dicarboxy-3,3′-bis(naphthylamido)-diphenylmethanone, 3,3′-dicarboxy-4,4′-bis(4-biphenylamido) diphenylmethane, 3,3′-dicarboxy-4,4′-bis(3-nitrophenylamido)diphenylmethane, RNase Inhibitor-Murine (NEB M0314), Protector RNase Inhibitor (Roche, RNAINH-RO)B-mercaptoethanol, RNAsecure (Thermo Fisher Scientific, AM7005), dithiothreitol (DTT), dithioerythritol (DTE), Tris (2-carboxyethyl) phosphine (TCEP) glutathione and cysteine.

In some embodiments, the nuclease inhibitor is present in a concentration of about 0.01 Unit/μl to about 1 Unit/μl.

In some embodiments, the nuclease inhibitor is present in a concentration of about 0.03 Unit/μl to about 0.75 Unit/μl.

In some embodiments, the nuclease inhibitor is present in a concentration of about 0.15 Unit/μl.

In some embodiments, the decoy oligonucleotide is present in a concentration of about 0.01 μg/μl to about 1 μg/μl.

In some embodiments, the decoy oligonucleotide is present in a concentration of about 0.01 μg/μl to about 0.25 μg/μl.

In some embodiments, the decoy oligonucleotide is present in a concentration of about 0.05 μg/μl.

In some embodiments, the composition further comprises an agent capable of amplifying a nucleic acid.

In some embodiments, the agent capable of amplifying a nucleic acid comprises a DNA polymerase.

In some embodiments, the composition further comprises a reverse transcriptase.

In some embodiments, the agent capable of amplifying a nucleic acid is present in a concentration of about 0.1 Unit/μl to about 5 Unit/μl.

In some embodiments, the agent capable of amplifying a nucleic acid is present in a concentration of about 0.1 Unit/μl to about 3 Unit/μl.

In some embodiments, the agent capable of amplifying a nucleic acid is present in a concentration of about 0.32 Unit/μl.

In some embodiments, the reverse transcriptase is present in a concentration of about 0.1 Unit/μl to about 5 Unit/μl.

In some embodiments, the reverse transcriptase is present in a concentration of about 0.1 Unit/μl to about 3 Unit/μl.

In some embodiments, the reverse transcriptase is present in a concentration of about 0.3 Unit/μl.

In some embodiments, the composition further comprises a buffer.

In some embodiments, the buffer comprises an isothermal amplification buffer.

In some embodiments, the composition further comprises a lysing agent.

In some embodiments, the lysing agent comprises a surfactant.

In some embodiments, the lysing agent comprises a polysorbate surfactant.

In some embodiments, the lysing agent comprises a Tween.

In some embodiments, the lysing agent comprises a Tween 20.

In some embodiments, the lysing agent is present in a concentration of about 0.01% (v/v) to about 1.5% (v/v).

In some embodiments, the lysing agent is present in a concentration of about 0.01% (v/v) to about 1% (v/v).

In some embodiments, the lysing agent is present in a concentration of about 0.1% (v/v).

In some embodiments, the composition further comprises dNTPs.

In some embodiments, each dNTP is present in a concentration of about 0.5 mM to about 5 mM.

In some embodiments, each dNTP is present in a concentration of about 1.4 mM.

In some embodiments, the composition further comprises one or more primers specific for the target nucleic acid.

In some embodiments, the one or more primers are suitable for amplifying the target nucleic acid in an isothermal amplification.

In some embodiments, the isothermal amplification comprises NASBA (nucleic acid sequence-based amplification), loop-mediated isothermal amplification (LAMP), recombinase polymerase amplification (RPA), and/or helicase-dependent amplification (HDA).

In some embodiments, the primer does not contain 5 or more consecutive identical single nucleotides.

In some embodiments, the primer does not contain 4 or more consecutive dinucleotide repeats.

In some embodiments, the primer contains at most one fragment of 4 consecutive identical single nucleotides.

In some embodiments, the primer contains at most two fragments of 3 consecutive identical single nucleotides.

In some embodiments, the primer does not form any secondary structure with a ΔG lower than about −4 kcal/mol.

In some embodiments, the primer does not comprise a 3′ end region having 6 or more nucleotides that are reverse complementary to a sequence within the same primer or within another primer of the more primers.

In some embodiments, the primer does not comprise a 3′ end region having 6 or more consecutive nucleotides that are reverse complementary to a sequence within the same primer or within another primer of the more primers.

In some embodiments, the target nucleic acid is a pathogen-associated nucleic acid.

In some embodiments, the pathogen is a virus.

In some embodiments, the target nucleic acid is a virus RNA.

In some embodiments, the target nucleic acid is a SARS-COV-2-associated nucleic acid, and wherein the primer comprises a nucleic acid sequence that is 1) at least 98% homologous to the corresponding sequence of each known SARS-COV-2 strain, or 2) complementary to a nucleic acid sequence that is at least 98% homologous to the corresponding sequence of each known SARS-COV-2 strain.

In some embodiments, the target nucleic acid is a SARS-COV-2-associated nucleic acid, and wherein the last 5 nucleotides from the extension start position of each primer are 100% homologous to the corresponding sequence of each known SARS-COV-2 strain, or 2) are complementary to a nucleic acid sequence that is 100% homologous to the corresponding sequence of each known SARS-CoV-2 strain.

In some embodiments, the target nucleic acid is a SARS-COV-2-associated nucleic acid, and the one or more primers comprise primers 1-6, the primer 1 comprises a nucleotide sequence as set forth in any of SEQ ID NO.3, SEQ ID NO.13, SEQ ID NO.33, SEQ ID NO.45, SEQ ID NO.63, SEQ ID NO.73, SEQ ID NO.83, SEQ ID NO.93, SEQ ID NO.106, SEQ ID NO.116, SEQ ID NO.126, SEQ ID NO.136, SEQ ID NO.146 and SEQ ID NO.156, the primer 2 comprises a nucleotide sequence as set forth in any of SEQ ID NO.4, SEQ ID NO.14, SEQ ID NO.34, SEQ ID NO.46, SEQ ID NO.64, SEQ ID NO.74, SEQ ID NO.84, SEQ ID NO.94, SEQ ID NO.107, SEQ ID NO.117, SEQ ID NO.127, SEQ ID NO.137, SEQ ID NO.147 and SEQ ID NO.157, the primer 3 comprises a nucleotide sequence as set forth in any of SEQ ID NO.1, SEQ ID NO.11, SEQ ID NO.31, SEQ ID NO.41, SEQ ID NO.43, SEQ ID NO.55, SEQ ID NO. 61, SEQ ID NO. 71, SEQ ID NO. 81, SEQ ID NO.91, SEQ ID NO.101, SEQ ID NO.104, SEQ ID NO.114, SEQ ID NO.124, SEQ ID NO.134, SEQ ID NO.144, SEQ ID NO.154 AND SEQ 1.154 and SEQ ID NO.163, the primer 4 comprises a nucleotide sequence as set forth in any of SEQ ID NO.2, SEQ ID NO.12, SEQ ID NO.32, SEQ ID NO.42, SEQ ID NO.44, SEQ ID NO.56, SEQ ID NO.62, SEQ ID NO.72, SEQ ID NO.82, SEQ ID NO.92, SEQ ID NO.102, SEQ ID NO.105, SEQ ID NO.115, SEQ ID NO.125, SEQ ID NO.135, SEQ ID NO.145 and SEQ ID NO.155.155, the primer 5 comprises a nucleotide sequence as set forth in any of SEQ ID NO.5, SEQ ID NO.15, SEQ ID NO.35, SEQ ID NO.47, SEQ ID NO.53, SEQ ID NO.55, SEQ ID NO.57, SEQ ID NO.59, SEQ ID NO.65, SEQ ID NO.75, SEQ ID NO.85, SEQ ID NO.95, SEQ ID NO.103, SEQ ID NO.108, SEQ ID NO.118, SEQ ID NO.128, SEQ ID NO.138, SEQ ID NO.148 SEQ ID NO.148 and SEQ ID NO.158, and the primer 6 comprises a nucleotide sequence as set forth in any of SEQ ID NO.6, SEQ ID NO.16, SEQ ID NO.36, SEQ ID NO.48, SEQ ID NO.54, SEQ ID NO.56, SEQ ID NO.58, SEQ ID NO.60, SEQ ID NO.66, SEQ ID NO.76, SEQ ID NO.86, SEQ ID NO.96, SEQ ID NO.109, SEQ ID NO.119, SEQ ID NO.129, SEQ ID NO.139 and SEQ ID NO.149.

In some embodiments, each of the one or more primers is present in a concentration of about 0.05 μM to about 5 μM.

In some embodiments, the primer 1 is present in a concentration of about 1 μM to about 2 μM, the primer 2 is present in a concentration of about 1 μM to about 2 μM, the primer 3 is present in a concentration of about 0.1 μM to about 0.5 μM, the primer 4 is present in a concentration of about 0.1 μM to about 0.5 μM, the primer 5 is present in a concentration of about 0.3 μM to about 0.8 μM, and the primer 6 is present in a concentration of about 0.3 μM to about 0.8 μM.

In some embodiments, the sample is a biological sample.

In some embodiments, the sample is selected from the group consisting of: nasopharyngeal swab, blood, serum, urine, saliva, tissue, cell, organ, a fraction or portion thereof, and any combination of the foregoing.

In some embodiments, the sample is a crude sample derived directly from a subject without being processed.

In some embodiments, the target nucleic acid in the sample has not been subjected to amplification and/or purification.

In some embodiments, the composition further comprises an electroactive indicator.

In some embodiments, the electroactive indicator comprises methylene blue.

In another aspect, the present application provides an isolated oligonucleotide, comprising a sequence as set forth in any of SEQ ID Nos. 1-20, 31-183.

In some embodiments, the isolated oligonucleotide is capable of specifically binding to a SARS-COV-2-associated nucleic acid.

In another aspect, the present application provides a primer, which is capable of specifically amplifying a SARS-COV-2—associated nucleic acid in an isothermal amplification.

In some embodiments, the isothermal amplification comprises NASBA (nucleic acid sequence-based amplification), loop-mediated isothermal amplification (LAMP), recombinase polymerase amplification (RPA), and/or helicase-dependent amplification (HDA).

In some embodiments, the primer does not contain 4 or more consecutive identical single nucleotides.

In some embodiments, the primer does not contain 4 or more consecutive dinucleotide repeats.

In some embodiments, the primer contains at most one fragment of 4 consecutive identical single nucleotides.

In some embodiments, the primer contains at most two fragments of 3 consecutive identical single nucleotides.

In some embodiments, the primer does not form any secondary structure with a ΔG lower than about −4 kcal/mol.

In some embodiments, the primer does not comprise a 3′ end region having 6 or more nucleotides that are reverse complementary to a sequence within the same primer or within another primer of the more primers.

In some embodiments, the primer does not comprise a 3′ end region having 6 or more consecutive nucleotides that are reverse complementary to a sequence within the same primer or within another primer of the more primers.

In some embodiments, the primer comprises a nucleic acid sequence that is 1) at least 98% homologous to the corresponding sequence of each known SARS-COV-2 strain, or 2) complementary to a nucleic acid sequence that is at least 98% homologous to the corresponding sequence of each known SARS-COV-2 strain.

In some embodiments, the last 5 nucleotides from the extension start position of each primer are 100% homologous to the corresponding sequence of each known SARS-COV-2 strain, or 2) are complementary to a nucleic acid sequence that is 100% homologous to the corresponding sequence of each known SARS-COV-2 strain.

In some embodiments, the one or more primers comprise primers 1-6, the primer 1 comprises a nucleotide sequence as set forth in any of SEQ ID NO.3, SEQ ID NO.13, SEQ ID NO.33, SEQ ID NO.45, SEQ ID NO.63, SEQ ID NO.73, SEQ ID NO.83, SEQ ID NO.93, SEQ ID NO.106, SEQ ID NO.116, SEQ ID NO.126, SEQ ID NO.136, SEQ ID NO.146 and SEQ ID NO.156, the primer 2 comprises a nucleotide sequence as set forth in any of SEQ ID NO.4, SEQ ID NO.14, SEQ ID NO.34, SEQ ID NO.46, SEQ ID NO.64, SEQ ID NO.74, SEQ ID NO.84, SEQ ID NO.94, SEQ ID NO.107, SEQ ID NO.117, SEQ ID NO.127, SEQ ID NO.137, SEQ ID NO.147 and SEQ ID NO.157, the primer 3 comprises a nucleotide sequence as set forth in any of SEQ ID NO.1, SEQ ID NO.11, SEQ ID NO.31, SEQ ID NO.41, SEQ ID NO.43, SEQ ID NO.55, SEQ ID NO. 61, SEQ ID NO. 71, SEQ ID NO. 81, SEQ ID NO.91, SEQ ID NO.101, SEQ ID NO.104, SEQ ID NO.114, SEQ ID NO.124, SEQ ID NO.134, SEQ ID NO.144, SEQ ID NO.154 AND SEQ 1.154 and SEQ ID NO.163, the primer 4 comprises a nucleotide sequence as set forth in any of SEQ ID NO.2, SEQ ID NO.12, SEQ ID NO.32, SEQ ID NO.42, SEQ ID NO.44, SEQ ID NO.56, SEQ ID NO.62, SEQ ID NO.72, SEQ ID NO.82, SEQ ID NO.92, SEQ ID NO.102, SEQ ID NO.105, SEQ ID NO.115, SEQ ID NO.125, SEQ ID NO.135, SEQ ID NO.145 and SEQ ID NO.155.155, the primer 5 comprises a nucleotide sequence as set forth in any of SEQ ID NO.5, SEQ ID NO.15, SEQ ID NO.35, SEQ ID NO.47, SEQ ID NO.53, SEQ ID NO.55, SEQ ID NO.57, SEQ ID NO.59, SEQ ID NO.65, SEQ ID NO.75, SEQ ID NO.85, SEQ ID NO.95, SEQ ID NO.103, SEQ ID NO.108, SEQ ID NO.118, SEQ ID NO.128, SEQ ID NO.138, SEQ ID NO.148 SEQ ID NO.148 and SEQ ID NO.158, and the primer 6 comprises a nucleotide sequence as set forth in any of SEQ ID NO.6, SEQ ID NO.16, SEQ ID NO.36, SEQ ID NO.48, SEQ ID NO.54, SEQ ID NO.56, SEQ ID NO.58, SEQ ID NO.60, SEQ ID NO.66, SEQ ID NO.76, SEQ ID NO.86, SEQ ID NO.96, SEQ ID NO.109, SEQ ID NO.119, SEQ ID NO.129, SEQ ID NO.139 and SEQ ID NO.149.

In some embodiments, each of the one or more primers is present in a concentration of about 0.05 μM to about 5 μM.

In some embodiments, the primer 1 is present in a concentration of about 1 μM to about 2 μM, the primer 2 is present in a concentration of about luM to about 2 μM, the primer 3 is present in a concentration of about 0.1 μM to about 0.5 μM, the primer 4 is present in a concentration of about 0.1 μM to about 0.5 μM, the primer 5 is present in a concentration of about 0.3 μM to about 0.8 μM, and the primer 6 is present in a concentration of about 0.3 μM to about 0.8 μM.

In another aspect, the present application provides a primer set, comprising primers 1-6, the primer 1 comprises a nucleotide sequence as set forth in any of SEQ ID NO.3, SEQ ID NO.13, SEQ ID NO.33, SEQ ID NO.45, SEQ ID NO.63, SEQ ID NO.73, SEQ ID NO.83, SEQ ID NO.93, SEQ ID NO.106, SEQ ID NO.116, SEQ ID NO.126, SEQ ID NO.136, SEQ ID NO.146 and SEQ ID NO.156, the primer 2 comprises a nucleotide sequence as set forth in any of SEQ ID NO.4, SEQ ID NO.14, SEQ ID NO.34, SEQ ID NO.46, SEQ ID NO.64, SEQ ID NO.74, SEQ ID NO.84, SEQ ID NO.94, SEQ ID NO.107, SEQ ID NO.117, SEQ ID NO.127, SEQ ID NO.137, SEQ ID NO.147 and SEQ ID NO.157, the primer 3 comprises a nucleotide sequence as set forth in any of SEQ ID NO.1, SEQ ID NO.11, SEQ ID NO.31, SEQ ID NO.41, SEQ ID NO.43, SEQ ID NO.55, SEQ ID NO. 61, SEQ ID NO. 71, SEQ ID NO. 81, SEQ ID NO.91, SEQ ID NO.101, SEQ ID NO.104, SEQ ID NO.114, SEQ ID NO.124, SEQ ID NO.134, SEQ ID NO.144, SEQ ID NO.154 AND SEQ 1.154 and SEQ ID NO.163, the primer 4 comprises a nucleotide sequence as set forth in any of SEQ ID NO.2, SEQ ID NO.12, SEQ ID NO.32, SEQ ID NO.42, SEQ ID NO.44, SEQ ID NO.56, SEQ ID NO.62, SEQ ID NO.72, SEQ ID NO.82, SEQ ID NO.92, SEQ ID NO.102, SEQ ID NO.105, SEQ ID NO.115, SEQ ID NO.125, SEQ ID NO.135, SEQ ID NO.145 and SEQ ID NO.155.155, the primer 5 comprises a nucleotide sequence as set forth in any of SEQ ID NO.5, SEQ ID NO.15, SEQ ID NO.35, SEQ ID NO.47, SEQ ID NO.53, SEQ ID NO.55, SEQ ID NO.57, SEQ ID NO.59, SEQ ID NO.65, SEQ ID NO.75, SEQ ID NO.85, SEQ ID NO.95, SEQ ID NO.103, SEQ ID NO.108, SEQ ID NO.118, SEQ ID NO.128, SEQ ID NO.138, SEQ ID NO.148 SEQ ID NO.148 and SEQ ID NO.158, and the primer 6 comprises a nucleotide sequence as set forth in any of SEQ ID NO.6, SEQ ID NO.16, SEQ ID NO.36, SEQ ID NO.48, SEQ ID NO.54, SEQ ID NO.56, SEQ ID NO.58, SEQ ID NO.60, SEQ ID NO.66, SEQ ID NO.76, SEQ ID NO.86, SEQ ID NO.96, SEQ ID NO.109, SEQ ID NO.119, SEQ ID NO.129, SEQ ID NO.139 and SEQ ID NO.149.

In another aspect, the present application provides a kit for detecting a target nucleic acid in a sample, comprising the composition of the present application, the isolated oligonucleotide of the present application, the primers of the present application, and/or the primer set of the present application.

In another aspect, the present application provides a use of the composition of the present application, the isolated oligonucleotide of the present application, the primers of the present application, and/or the primer set of the present application. in the manufacture of an agent for detecting a target nucleic acid in a sample.

In some embodiments, the sample is selected from the group consisting of: nasopharyngeal swab, blood, serum, urine, saliva, tissue, cell, organ, a fraction or portion thereof, and any combination of the foregoing.

In some embodiments, the sample is a crude sample derived directly from a subject without being processed.

In some embodiments, the target nucleic acid is a pathogen-associated nucleic acid.

In some embodiments, the target nucleic acid is a SARS-COV-2-associated nucleic acid.

In another aspect, the present application provides a system for detecting a target nucleic acid in a sample, comprising a sample analyzing module and a display module, wherein the sample analyzing module and the display module are informationally connected, the sample analyzing module comprise the composition of the present application, the isolated oligonucleotide of the present application, the primers of the present application, and/or the primer set of the present application, and the display module is configured to indicate a presence and/or amount of the target nucleic acid in the sample based on an information generated by the sample analyzing module.

In another aspect, the present application provides a method for detecting a target nucleic acid in a sample, comprising administering the composition of the present application, the isolated oligonucleotide of the present application, the primers of the present application, and/or the primer set of the present application.

In another aspect, the present application provides a method, comprising: contacting a sample suspected to contain a target nucleic acid with the composition of any one of claims ______ to obtain a one pot reaction mixture.

In some embodiments, the sample is selected from the group consisting of: nasopharyngeal swab, blood, serum, urine, saliva, tissue, cell, organ, a fraction or portion thereof, and any combination of the foregoing.

In some embodiments, the sample is a crude sample derived directly from a subject without being processed.

In some embodiments, the sample has not been subjected to nucleic acid purification, cell lysis, and/or nucleic acid amplification.

In some embodiments, the method further comprises: subjecting the one pot reaction mixture to nucleic acid amplification.

In some embodiments, the nucleic acid amplification comprises an isothermal nucleic acid amplification reaction.

In some embodiments, the nucleic acid amplification comprises NASBA, LAMP, and/or RPA.

In some embodiments, the nucleic acid amplification is conducted at a single temperature of about 55° C. to about 70° C.

In some embodiments, before and/or during the nucleic acid amplification, an electric potential is applied to the reaction mixture, and a presence and/or an amount of an amplification product is indicated by a change of an electric signal and/or a fluorescent signal generated during and/or after the nucleic acid amplification.

In some embodiments, the electric signal is a redox current, and a decrease of the redox current indicates a presence and/or increase of the amplification product.

Additional aspects and advantages of the present application will become readily apparent to those skilled in this art from the following detailed description, wherein only illustrative embodiments of the present application are shown and described. As will be realized, the present application is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are employed, and the accompanying drawings (also “figure” and “FIG.” herein), of which:

FIGS. 1A-1C illustrate exemplary oligonucleotides of the present application.

FIG. 2 illustrates exemplary measurement of SARS-COV-2 from the crude samples within 30 minutes by the composition and/or the system of present application.

FIGS. 3A-3B illustrate the determination of the limit of detection (LOD) for the composition and/or the system of the present application.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used herein, the singular forms “a”, “and”, and “the” include plural referents unless the context clearly dictates otherwise. Thus, for example, reference to “a particle” includes a plurality of such particles and reference to “the sequence” includes reference to one or more the sequences and equivalents thereof known to those skilled in the art, and so forth.

As used herein, the term “about” generally refers to within 10% of a stated value. For example, the about may refer to within 10% (for example, within about 9%, within about 8%, within about 7%, within about 6%, within about 5%, within about 4%, within about 3%, within about 2%, or within about 1%) of a stated concentration range. For example, the about may refer to within 10% (for example, within about 9%, within about 8%, within about 7%, within about 6%, within about 5%, within about 4%, within about 3%, within about 2%, or within about 1%) of a stated time frame.

Where a range of values (e.g., a numerical range) is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

As used herein, the term “a target nucleic acid” generally refers to a nucleic acid of interest to be detected. In the present application, the target nucleic acid may be originated from a pathogen, and/or a pathogen-associated nucleic acid.

As used herein, the term “decoy oligonucleotide” generally refers to an oligonucleotide used to affect or influence a reaction of interest. For example, the decoy oligonucleotide may play a role in preventing a target nucleic acid from being degraded in a reaction (e.g., a detection reaction). In the present application, the decoy oligonucleotide may also be used to enhance certain reactions, for example by affecting the secondary structure of a nucleotide target, or by inhibiting off-target reactions. In the present application, the decoy oligonucleotide may play a role in competitively binding to a nuclease (For example, a DNase, or an RNase). For example, the decoy oligonucleotide may compete with a target nucleic acid for binding to a nuclease. For example, the decoy oligonucleotide may be different from the target nucleic acid in the present application. For example, the decoy oligonucleotide may comprise synthetic or naturally produced nucleic acid molecules. For example, said decoy oligonucleotide may comprise a natural tRNA.

As used herein, the term “a non-target nucleic acid molecule” generally refers to a nucleic acid molecule which is structurally and/or functionally substantially different from the target nucleic acid molecule. Said non-target nucleic acid molecule may play a role in preventing a target nucleic acid from being degraded in a reaction (e.g., a detection reaction). For example, the non-target nucleic acid molecule may not be originated from a pathogen, nor be a pathogen-associated nucleic acid. For example, the non-target nucleic acid molecule may originate from a different pathogen compared to the target nucleic acid molecule. For example, the non-target nucleic acid molecule and the target nucleic acid molecule may originate from different parts of a pathogen. In present application, the non-target nucleic acid molecule may be tRNA. For example, said tRNA may be naturally produced. For example, said tRNA may be originated from a yeast.

As used herein, the term “specifically binding” generally refers to specifically binding to one protein and does not specifically bind other protein. In the present application, the decoy oligonucleotide (for example, the non-target nucleic acid molecule) may bind to a nuclease with a high affinity. For example, the decoy oligonucleotide may bind to the nuclease with an equilibrium dissociation constant (KD) that is the at least one hundred folds less than the KD of the decoy oligonucleotide binding to a target that is not a nuclease. For example, the decoy oligonucleotide may bind to the nuclease with an KD that is the at least about 150 folds, about 200 folds, about 250 folds, about 300 folds, about 400 folds, about 500 folds, about 1000 folds, about 5000 folds, about 10000 folds or more, less than the Kp of the decoy oligonucleotide binding to a target that is not a nuclease.

As used herein, the term “RNase” generally refers to Ribonuclease, which is a type of nuclease that catalyzes the degradation of RNA into smaller fragments. There are various RNase for a person skilled in the art to choose. For example, the RNase may comprise RNase A, RNase B and/or RNase C. In the present application, the Ribonuclease A or RNase A is an endoribonuclease, which is an enzyme specific for single-stranded RNAs. It cleaves the 3′-end of unpaired C and U residues, ultimately forming a 3′-phosphorylated product via a 2′,3′-cyclic monophosphate intermediate. In the present application, the Ribonuclease B (RNase B) is a form of the enzyme RNase A that has an added glycoprotein with N-linked carbohydrates at Asn-34, which means that the carbohydrate is attached at the nitrogen of the Aspargine-34 side chain. This small change may allow RNase B to hydrolyze double-stranded RNA at ionic strengths where RNase A has no activity. In the present application, the RNase C or RNase III, is a type of ribonuclease that recognizes dsRNA and cleaves it at specific targeted locations to transform them into mature RNAs. The RNase C may play a role in RNA precursor synthesis and RNA silencing.

As used herein, the term “tRNA” generally refers to a type of RNA molecule that helps decode a messenger RNA (mRNA) sequence into a protein. The tRNA may comprise the following components: a 5′terminal phosphate group; a acceptor stem made by the base pairing of the 5′-terminal nucleotide with the 3′-terminal nucleotide; a CCA tail at the 3′ terminal; a D arm, comprising dihydrouridine and forming in a loop; a anticodon arm comprising the anticodon and a T arm comprising a sequence TYΨC and Ψ is pseudouridine.

As used herein, the term “RNase inhibitor” generally refers to a compound that is capable of inhibiting an RNase activity. In the present application, the RNase inhibitor may be able to inactivate ribonuclease enzymes, which degrade RNA. In the present application, the RNase inhibitor may also be able to inhibit other enzymes such as reverse transcriptase and/or DNase I.

As used herein, the term “protein-based RNase inhibitor” generally refers to a protein and or polypeptide capable of specifically binding to a RNase and inhibiting one or more activities (such as RNA-degrading activity) of the RNase. In the present application, the protein-based RNase inhibitor may be able to bind to the RNase (for example, RNase A) to form noncovalent complexes that are enzymatically inactive. In the present application, the protein-based RNase inhibitor may be able to have a narrow spectrum of specificity. In the present application, the protein-based RNase inhibitor may be able to be easily denatured. In the present application, the protein-based RNase inhibitor may be able to comprise an RNase inhibitor protein (RIP), a protease, a tyrosine-glutamate copolymer, actin, or RraA. The RIP may be obtained from a human, a chimpanzee, a rat, a mouse, a pig, or a yeast. The RIP may also be obtained by recombinant means and derivatives thereof. In the present application, the protein-based RNase inhibitor may be also be a protease that irreversibly inactivates the RNase by cleaving the nuclease into peptide fragments. For example, the protein-based RNase inhibitor may be able to inhibit the activity of different RNases while not inhibit the activity of a nucleic acid polymerase. For example, the protein-based RNase inhibitor may comprise proteinase K, subtilisin, other alkaline proteases, acid proteases (e.g., pepsin), and/or pancreatic proteases (e.g., elastase, trypsin, and chymotrypsin).

As used herein, the term “chemical-based RNase inhibitor” generally refers to a chemical compound capable of specifically binding to a RNase and inhibiting one or more activities (such as RNA-degrading activity) of the RNase. In the present application, the chemical-based RNase inhibitor may be able to specifically inhibit the activity of RNase. In the present application, the chemical-based RNase inhibitor may also be able to inhibit other enzymes such as reverse transcriptase and DNase I. In the present application, the chemical-based RNase inhibitor may comprise sodium dodecylsulfate (SDS), Ethylene Diamine Tetraacetic Acid (EDTA), heparin, hydroxylamine-oxygen-cupric ion, bentonite and ammonium sulfate.

As used herein, the term “activity of RNase A, RNase B and/or RNase C” generally refers to one or more activities (such as RNA-degrading activity) of the RNase A, RNase B and/or RNase C. For example, the activity may comprise RNA-binding activity, RNA-degrading activity, and/or RNA-removing activity.

As used herein, the term “substantially inhibit” generally refers to a substantial inhibition of one or more activities (for example, an amplification activity) of a nucleic acid polymerase (for example, a DNA polymerase, and/or a reverse transcriptase). For example, the RNase inhibitor of the present application may be able to bind a nucleic acid polymerase with a K_{D} which may be at least one hundred folds less than the K_{D} of a nucleic acid polymerase inhibitor (for example, with an K_{D} that is the at least about 150 folds, about 200 folds, about 250 folds, about 300 folds, about 400 folds, about 500 folds, about 1000 folds, about 5000 folds, about 10000 folds or more, less than the K_{D} of the nucleic acid polymerase inhibitor). For example, the RNase inhibitor may be able not to substantially affect or influence the formation of DNA, and/or the formation of RNA. For example, the RNase inhibitor may be able to decrease the formation of new copies of DNA and/or RNA.

As used herein, the term “isothermal amplification buffer” generally refers to a buffer which is needed in an isothermal amplification. In the present application, the isothermal amplification may be able to provide detection of a nucleic acid target sequence in a streamlined, exponential manner, and are not limited by the constraint of thermal cycling. The isothermal amplification may rely on a polymerase with strand-displacement activity. There are various kinds of the isothermal amplification, for example, Loop-mediated isothermal amplification (LAMP), Whole genome amplification (WGA), NASBA, HDA, RCA, MDA, WGA and/or RPA. In the present application, the LAMP may be a rugged, low-cost method for specific DNA detection, with a visual readout. The LAMP may not require a dedicated thermocycler. And the LAMP may be based on using a set of six primers, and result in the improved specificity and sensitivity. In the present application, the isothermal amplification buffer may comprise components necessary for conducting the isothermal amplification, for example, the isothermal amplification buffer may comprise 1×Isothermal Amplification Buffer II (NEB).

As used herein, the term “lysing agent” generally refers to an agent being capable of cell lysis. In the present application, the lysing agent may be able to help lysis of a cell from a sample. For example, the lysing agent may be able to help releasing the target nucleic acid from a sample. For example, the lysing agent may comprise an enzyme, a detergent and/or a chaotropic agent.

As used herein, the term “polysorbate surfactant” generally refers to a polysorbate-type nonionic surfactant. For example, said polysorbate surfactant may comprise peroxides, which can oxidize amino acid residues that are prone to oxidation (Kishore, Kiese, et al., 2011; Kishore, Pappenberger, et al., 2011). For example, said polysorbate surfactant may comprise Polysorbate 20, Polysorbate 60 and/or Polysorbate 80.

As used herein, the term “LAMP” generally refers to a method that amplifies DNA under isothermal conditions (for example, at a constant temperature of about 60-about 65° C). The LAMP may be able to amplify DNA with high efficiency and specificity. In the present application, the LAMP may be able to employ a DNA polymerase, and either two or three sets of primers to amplify a target sequence. For example, four different primers may be able to amplify 6 distinct regions on the target sequence. And an additional pair of primers (i.e. loop primers) may be able to further accelerate the amplification. In the present application, the LAMP may be able to be conducted in a tube.

As used herein, the term “secondary structure” generally refers to a spatial or steric configuration formed by a molecule (such as a nucleic acid molecule). For example, the secondary structure may be formed through interactions among nucleotides and/or nucleic bases within a nucleic acid molecule. A DNA molecule may fold into various inter- and intramolecular secondary structures. For example, the secondary structure may comprise a right-handed double helical structure of B-form DNA (B-DNA) and a non-B-form secondary structures, which may comprise a G-quadruplex structures (G4 structures), a Z-DNA, a cruciforms and a triplexe (Matthew L. Bochman et al., Nat Rev Genet. 2012 Nov; 13(11): 770-780).

As used herein, the term “corresponding sequence” generally refers to a sequence originated from a subject is corresponding to another sequence originated from another different subject when these two sequences being aligned. For example, the subject may be a strain of a pathogen, and another different subject may be another strain of the same species of the pathogen.

As used herein, the term “crude sample” generally refers to any material which is directly derived and/or collected from a subject. In the present application, the crude sample may comprise, or potentially comprise, which could be contained, infected, or contaminated by the presence of a target nucleic acid. In present application, the crude sample may be untreated nor be unprocessed. For example, the crude sample may have not been gone through purification while the purification enhances the purity and/or relative quantity of the target nucleic acid of the present application. For example, the crude sample may not have been lysis while the lysis help release the target nucleic acid of the present application from a cell in the crude sample. For example, the crude sample may not have been gone through enrichment while the enrichment enhances the relative concentration of the target nucleic acid of the present application.

As used herein, the term “processed” generally refers to any treatment and/or any process administrated to the crude sample of the present application before the crude sample contacting with the composition of the present application. For example, the treatment and/or the process may comprise any measurement to enhance the quantity and/or the quality of the target nucleic acid in the crude sample. In the present application, the treatment and/or the process may comprise enrichment, amplification concentrating and/or purification.

As used herein, the term “electroactive indicator” generally refers to an equipment which may be able to detect a hybridization event through detecting signals. For example, the signal may comprise oxidation signal and/or reduction signal. In the present application, the electroactive indicator may be able to be used for DNA hybridization detection. In the present application, the electroactive indicator may comprise electroactive compound, for example, metal complex such as ruthenium bipyridine, cobalt phenanthroline and cobalt bipyridine, and organic dyes such as methylene blue, nile blue and brilliant cresyl blue.

As used herein, the term “informationally connected” generally refers to the sample analyzing module of present application and the display module of the present application are connected via a shared information. In the present application, the information may comprise whether the target nucleic acid is detected by the sample analyzing module. For example, if the target nucleic acid is detected by the sample analyzing module, then the sample analyzing module may be able to send a signal about the target nucleic acid “positive” to the display module, and the display module may show the positive result to the subject. However, if the target nucleic acid is not detected by the sample analyzing module, then the sample analyzing module may be able to send a signal about the target nucleic acid “negative” to the display module, and the display module may show the negative result to the subject.

As used herein, the term “one pot reaction mixture” generally refers to a mixture ready to go through a one-pot reaction. In the present application, the one-pot reaction mixture may comprise the composition of the present application. For example, the one-pot reaction mixture may comprise the crude sample of the present application. For example, in the one pot reaction mixture (for example, sealed in a tube), the target nucleic acid of the present application from the crude sample may be able to be detected by the composition of the present application in a single reactor.

As used herein, the term “stocking solution” generally refers to a mixture comprising the components of the composition of the present application. The stocking solution may be prepared for adding the sample (for example, the crude sample) later. The concentration of a component of the composition (for example, may be a decoy oligonucleotide) in the stocking solution may be higher than (for example, may be twice higher to) that of in the composition of present application. The stocking solution may be prepared for sale and/or for detection directly.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. This is intended to provide support for all such combinations.

### Nuclease Inhibitor and Decoy Oligonucleotide

In one aspect, the present application provides a composition for detecting a target nucleic acid in a sample, the composition comprising: a nuclease inhibitor and a decoy oligonucleotide.

In the present application, the decoy oligonucleotide may be a non-target nucleic acid molecule capable of specifically binding to a nuclease. For example, the decoy oligonucleotide may be capable of specifically binding to an RNase.

In the present application, the decoy oligonucleotide may comprise tRNAs. For example, the decoy oligonucleotide may be naturally originated. For example, the decoy oligonucleotide may comprise or consist of natural tRNAs, such as tRNAs derived from a natural origin/organism (e.g., a yeast).

For example, the decoy oligonucleotide may comprise R8759 (Sigma Aldrich). The R8759 is a ribonucleic acid transferred from baker's yeast (S. cerevisiae) and appeared as lyophilized powder.

In the present application, the nuclease inhibitor may comprise an RNase inhibitor.

In the present application, the RNase inhibitor may comprise a protein-based RNase inhibitor and/or a chemical-based RNase inhibitor.

In the present application, the RNase inhibitor may be capable of specifically inhibiting an activity of RNase A, RNase B and/or RNase C. For example, the RNase inhibitor may be capable of specifically inhibiting the RNA-binding activity, RNA-degrading activity, and/or RNA-removing activity of RNase A, RNase B and/or RNase C.

In the present application, the RNase inhibitor may be capable of reducing one or more disulfide bonds in an RNase.

In the present application, the RNase inhibitor may not substantially inhibit an activity of a nucleic acid polymerase. For example, the RNase inhibitor may not substantially inhibit an activity of a DNA polymerase, and/or an activity of a reverse transcriptase.

In the present application, the nuclease inhibitor may comprise one or more of the following: oligovinylsulfonic acid (OVA), aurintricarboxylic acid (ATA), aflatoxin, 2-nitro-5-thiocyanobenzoic acid, iodoacetate, N-bromosuccinimide, p-chloromercuribenzoate, diethyl pyrocarbonate, ethanol, formamide, guanidinium thiocyanate (GdnSCN), dinitrofluorobenzene, decanavanate, polyvinylsufonic acid, hydrobenzoinphosphate, phenylphosphate, putrescine, haloacetate, dinitrofluorobenzene, phenylglyoxal, bromopyruvic, hydroxylamine-oxygen-cupric ion, a vanadyl complex, 8-amino-5-(4′-hydroxy-biphenyl-4-ylazo)-naphthalene-2-sulfonate, 6-hydroxy-5-(2-hydroxy-3,5-dinitro-phenylazo)-naphthalene-2-sulfonate, 3,3′-dimethylbiphenyl-4,4′-bis(2-amino-naphthylazo-6-sulfonate), 4,4′-dicarboxy-3,3′-bis(naphthylamido)-diphenylmethanone, 3,3′-dicarboxy-4,4′-bis(4-biphenylamido) diphenylmethane, 3,3′-dicarboxy-4,4′-bis(3-nitrophenylamido)diphenylmethane, RNase Inhibitor-Murine (NEB M0314), Protector RNase Inhibitor (Roche, RNAINH-RO)B-mercaptoethanol, RNAsecure (Thermo Fisher Scientific, AM7005), dithiothreitol (DTT), dithioerythritol (DTE), Tris (2-carboxyethyl) phosphine (TCEP) glutathione and cysteine.

In present application, the concentration of a component (for example, the nuclease inhibitor) of the composition of present application may be illustrated as the “final concentration”. For example, the final concentration is the concentration after the sample is added and is ready for the detection.

In the present application, the nuclease inhibitor may present in a concentration of about 0.01 Unit/μl to about 1.5 Unit/μl. For example, the nuclease inhibitor may present in a concentration of about 0.01 Unit/μl to about 1 Unit/μl, about 0.03 Unit/μl to about 1 Unit/μl, about 0.15 Unit/μl to about 1 Unit/μl, about 0.03 Unit/μl to about 0.75 Unit/μl, about 0.15 Unit/μl to about 0.75 Unit/μl, about 0.15 Unit/μl to about 0.3 Unit/μl, about 0.06 Unit/μl to about 1.5 Unit/μl, about 0.3 Unit/μl to about 1.5 Unit/μl, or about 0.06 Unit/μl to about 0.3 Unit/μl.

In the present application, the nuclease inhibitor may present in a concentration of at least about 0.15 Unit/μl. For example, the nuclease inhibitor may present in a concentration of at least about 0.01 Unit/μl, at least about 0.03 Unit/μl, at least about 0.06 Unit/μl, at least about 0.15 Unit/μl, at least about 0.3 Unit/μl, at least about 0.75 Unit/μl, at least about 1 Unit/μl, or at least about 1.5 Unit/μl.

For example, in the composition of present application, the nuclease inhibitor may present in a concentration of about 0.01 Unit/μl to about 1 Unit/μl, or about 0.03 Unit/μl to about 0.75 Unit/μl. For example, the nuclease inhibitor may present in a concentration of at least about 0.03 Unit/μl, at least about 0.75 Unit/μl, or at least about 0.15 Unit/μl.

For example, in the stocking solution of present application, the nuclease inhibitor may present in a concentration of about 0.06 Unit/μl to about 1.5 Unit/μl. For example, the nuclease inhibitor may present in a concentration of at least about 0.06 Unit/μl, at least about 0.3 Unit/μl, or at least about 1.5 Unit/μl.

In the present application, the decoy oligonucleotide may present in a concentration of about 0.01 μg/μl to about 2 μg/μl. For example, the decoy oligonucleotide may present in a concentration of about 0.01 μg/μl to about 1 μg/μl, about 0.01 μg/μl to about 0.25 μg/μl, about 0.05 μg/μl to about 1 μg/μl, about 0.05 μg/μl to about 0.25 μg/μl, about 0.02 μg/μl to about 0.5 μg/μl, about 0.02 μg/μl to about 2 μg/μl, about 0.05 μg/μl to about 0.5 μg/μl, about 0.1 μg/μl to about 0.5 μg/μl.

In the present application, the decoy oligonucleotide may present in a concentration of at least about 0.05 μg/μl. For example, the decoy oligonucleotide may present in a concentration of at least about 0.01 μg/μl, at least about 0.02 μg/μl, at least about 0.05 μg/μl, at least about 0.1 μg/μl, at least about 0.25 μg/μl, at least about 0.5 μg/μl, at least about 1 μg/μl, at least about 2 μg/μl.

For example, in the composition of present application, the decoy oligonucleotide may present in a concentration of about 0.01 μg/μl to about lμg/μl, or about 0.01 μg/μl to about 0.25μg/μl. For example, the decoy oligonucleotide may present in a concentration of at least about 0.01 μg/μl, at least about 0.05 μg/μl, at least about 0.25 μg/μl, or at least about 1 μg/μl.

For example, in the stocking solution of present application, the decoy oligonucleotide may present in a concentration of about 0.02 μg/μl to about 2μg/μl, or about 0.02 μg/μl to about 0.5μg/μl. For example, the decoy oligonucleotide may present in a concentration of at least about 0.02 μg/μl, at least about 0.1 μg/μl, at least about 0.5 μg/μl, or at least about 2 μg/μl.

### Agents for Nucleic Acid Amplification

In the present application, the composition further may comprise an agent capable of amplifying a nucleic acid. For example, the composition further may comprise a nucleic acid polymerase.

In the present application, said amplification may comprise an isothermal amplification. For example, said isothermal amplification may be generally below the melting temperature (Tm; the temperature at which half of the potentially double-stranded molecules in a mixture are in a single-stranded, denatured state) of the predominant reaction product, i.e., generally about 90° C.or below, usually between about 50° C. and about 75° C.

In the present application, said isothermal amplification may comprise NASBA (nucleic acid sequence-based amplification), LAMP (loop-mediated isothermal amplification), strand displacement amplification (SDA), helicase-dependent amplification (HDA), nicking enzyme amplification reaction (NEAR), signal mediated amplification of RNA technology (SMART), rolling circle amplification (RCA), isothermal multiple displacement amplification (IMDA), single primer isothermal amplification (SPIA), polymerase spiral reaction (PSR) and/or RPA (recombinase polymerase amplification).

For example, said LAMP may be able to be conducted according to the manufacturer's instructions for Bst 2.0 DNA Polymerase (New England Biolabs). For example, said LAMP may be able to be conducted as the following in brief: a single reaction was prepared, the single reaction comprises Isothermal Amplification Buffer II, dNTP, FIP/BIP primers, F3/B3 primers, LF/LB primers, DNA Polymerase, DNA templates, and water. After mixing, the reaction was incubated at a temperature below the melting temperature for a period of time.

In the present application, the agent capable of amplifying a nucleic acid may comprise a DNA polymerase. The DNA polymerase may catalyze the synthesis of DNA molecules from nucleoside triphosphates, the molecular precursors of DNA.

For example, the DNA polymerase may comprise DNA polymerases having strand displacement activity, and such DNA polymerase may comprise those of phi29 DNA polymerase, DNA polymerase I, Klenow fragment, Klenow fragment (3′->5′ exo-), DNA polymerases isolated or derived from thermophilic organisms, e.g“ VENTR DNA Polymerase, 9 Nm DNA Polymerase, Therminator DNA Polymerase, Bacillus stearothermophilus (Bst) DNA polymerase (U.S. Pat. Nos. 5,874,282; 6,100,078, and 6,066,483, Riggs et al.), and the large fragment of Moloney murine leukemia virus (MMLV) reverse transcriptase (RT). For example, a Bst DNA polymerase may be modified to reduce, inhibit, inactivate or remove its 5′ exonuclease activity (i.e., 5′-exo-minus polymerase).

In the present application, the composition further may comprise a reverse transcriptase. The reverse transcriptase may be used to generate complementary DNA (cDNA) from an RNA template, during the process called reverse transcription.

For example, the reverse transcriptase may be able to have reverse transcriptase (RT) activity which catalyzes extension of a DNA complement from an RNA template (i.e., RNA directed DNA polymerase). For example, the reverse transcriptase may comprise MMLV RT and avian myeloblastosis virus (AMV) RT enzymes.

In the present application, the agent capable of amplifying a nucleic acid may present in a concentration of about 0.1 Unit/μl to about 10.0 Unit/μl. For example, the agent capable of amplifying a nucleic acid may present in a concentration of about 0.1 Unit/μl to about 5 Unit/μl, about 0.1 Unit/μl to about 3 Unit/μl, about 0.3 Unit/μl to about 3 Unit/μl, about 0.32 Unit/μl to about 3.0 Unit/μl, about 0.32 Unit/μl to about 5 Unit/μl, about 0.2 Unit/μl to about 6 Unit/μl, about 0.64 Unit/μl to about 6 Unit/μl or about 0.2 Unit/μl to about 0.64 Unit/μl.

In the present application, the agent capable of amplifying a nucleic acid may present in a concentration of at least about 0.32 Unit/μl. For example, the agent capable of amplifying a nucleic acid may present in a concentration of at least about 0.1 Unit/μl, at least about 0.2 Unit/μl, at least about 0.3 Unit/μl, at least about 0.32 Unit/μl, at least about 0.6 Unit/μl, at least about 0.64 Unit/μl, at least about 3 Unit/μl, at least about 5 Unit/μl or at least about 6.0 Unit/μl.

For example, in the composition of present application, the agent capable of amplifying a nucleic acid may present in a concentration of about 0.1 Unit/μl to about 5.0 Unit/μl, or about 0.1 Unit/μl to about 3.0 Unit/μl. For example, the agent capable of amplifying a nucleic acid may present in a concentration of at least about 0.1 Unit/μl, at least about 0.32 Unit/μl, at least about 3.0 Unit/μl or at least about 5.0 Unit/μl.

For example, in the stocking solution of present application, the agent capable of amplifying a nucleic acid may present in a concentration of about 0.2 Unit/μl to about 10.0 Unit/μl, or about 0.2 Unit/μl to about 6.0 Unit/μl. For example, the agent capable of amplifying a nucleic acid may present in a concentration of at least about 0.2 Unit/μl, at least about 0.64 Unit/μl or at least about 6.0 Unit/μl or at least about 10.0 Unit/μl.

In the present application, the reverse transcriptase may present in a concentration of about 0.1 Unit/μl to about 10.0 Unit/μl. For example, the reverse transcriptase may present in a concentration of about 0.1 Unit/μl to about 5 Unit/μl, about 0.1 Unit/μl to about 3 Unit/μl, about 0.3 Unit/μl to about 5 Unit/μl, about 0.3 Unit/μl to about 3.0 Unit/μl, about 0.2 Unit/μl to about 6 Unit/μl, about 0.6 Unit/μl to about 6 Unit/μl or about 0.2 Unit/μl to about 0.6 Unit/μl.

In the present application, the reverse transcriptase may present in a concentration of about 0.3 Unit/μl. For example, the reverse transcriptase may present in a concentration of at least about 0.1 Unit/μl, at least about 0.2 Unit/μl, at least about 0.3 Unit/μl, at least about 0.6 Unit/μl, at least about 0.64 Unit/μl, at least about 3 Unit/μl, at least about 5 Unit/μl or at least about 6.0 Unit/μl.

For example, in the composition of present application, the reverse transcriptase may present in a concentration of about 0.1 Unit/μl to about 5.0 Unit/μl, or about 0.1 Unit/μl to about 3.0 Unit/μl. For example, the reverse transcriptase may present in a concentration of at least about 0.1 Unit/μl, at least about 0.3 Unit/μl, at least about 3.0 Unit/μl or at least about 5.0 Unit/μl.

For example, in the stocking solution of present application, the reverse transcriptase may present in a concentration of about 0.2 Unit/μl to about 10.0 Unit/μl, or about 0.2 Unit/μl to about 6.0 Unit/μl. For example, the reverse transcriptase may present in a concentration of at least about 0.2 Unit/μl, at least about 0.6 Unit/μl, at least about 6.0 Unit/μl or at least about 10.0 Unit/μl.

In the present application, the composition further may comprise a buffer.

In the present application, the buffer may comprise an isothermal amplification buffer. For example, the isothermal amplification buffer may be able to provide a superior reaction condition for the isothermal amplification. For example, the isothermal amplification buffer may be optimized for use with Bst 2.0 DNA Polymerase and Bst 2.0 WarmStart™ DNA Polymerase.

In the present application, the composition further may comprise a lysing agent.

For example, the lysing agent may comprise a surfactant. In the present application, the lysing agent may comprise a polysorbate surfactant.

In the present application, the lysing agent may comprise a Tween.

For example, the lysing agent may comprise a Tween 20, a Tween 60, and/or a Tween 80.

In the present application, the lysing agent may present in a concentration of about 0.01% (v/v) to about 3.0% (v/v). For example, the lysing agent may present in a concentration of about 0.01% (v/v) to about 1.5% (v/v), about 0.01% (v/v) to about 1.0% (v/v), about 0.1% (v/v) to about 1.0% (v/v), about 0.01% (v/v) to about 0.1% (v/v), about 0.02% (v/v) to about 3.0% (v/v), about 0.02% (v/v) to about 2.0% (v/v), about 0.2% (v/v) to about 2.0% (v/v), or about 0.02% (v/v) to about 0.2% (v/v).

In the present application, the lysing agent may present in a concentration of at least about 0.1% (v/v). For example, the lysing agent may present in a concentration of at least about 0.01% (v/v), at least about 0.02% (v/v), at least about 0.1% (v/v), at least about 0.2% (v/v), at least about 1.0% (v/v), at least about 1.5% (v/v), at least about 2.0% (v/v) or at least about 3.0% (v/v).

For example, in the composition of present application, the lysing agent may present in a concentration of about 0.01% (v/v) to about 1.5% (v/v), about 0.01% (v/v) to about 1.0% (v/v), or about 0.1% (v/v) to about 1.0% (v/v). For example, the lysing agent may present in a concentration of at least about 0.01% (v/v), at least about 0.1% (v/v), or at least about 1.0% (v/v).

For example, in the stocking solution of present application, the lysing agent may present in a concentration of about 0.02% (v/v) to about 3.0% (v/v), about 0.02% (v/v) to about 2.0% (v/v), or about 0.2% (v/v) to about 2.0% (v/v). For example, the lysing agent may present in a concentration of at least about 0.02% (v/v), at least about 0.2% (v/v), or at least about 2.0% (v/v).

In the present application, the composition further may comprise dNTPs.

In the present application, the dNTP may comprise adenine (dATP), cytosine (dCTP), guanine (dGTP), and thymine (dTTP). DNA synthesis may require dATP, dGTP, dCTP, and dTTP as substrate. For example, the dNTP may be in a mix

In the present application, each dNTP may present in a concentration of about 0.5 mM to about 10 mM. For example, each dNTP may present in a concentration of about 0.5 mM to about 5 mM, about 1.4 mM to about 5 mM, about 0.5 mM to about 1.4 mM, about 1 mM to about 10 mM, about 2.8 mM to about 10 mM, or about 1 mM to about 2.8 mM.

In the present application, each dNTP may present in a concentration of at least about 1.4 mM. For example, each dNTP may present in a concentration of at least about 0.5 mM, at least about 1 mM, at least about 1.4 mM, at least about 2.8 mM, at least about 5 mM or at least about 10 mM.

For example, in the composition of present application, each dNTP may present in a concentration of about 0.5 mM to about 5 mM. For example, each dNTP may present in a concentration of at least about 0.5 mM, at least about 1.4 mM, or at least about 5 mM.

For example, in the stocking solution of present application, each dNTP may present in a concentration of about 1.0 mM to about 10 mM. For example, each dNTP may present in a concentration of at least about 1.0 mM, at least about 2.8 mM, or at least about 10 mM.

In the present application, said composition may comprise the decoy oligonucleotide of the present application, the nuclease inhibitor of the present application, the agent capable of amplifying a nucleic acid of the present application, the dNTP of the present application and the lysing agent of the present application.

In the present application, said composition may comprise the tRNA of the present application, the nuclease inhibitor of the present application, the agent capable of amplifying a nucleic acid of the present application, the dNTP of the present application and the lysing agent of the present application.

In the present application, said composition may comprise the tRNA of the present application, the RNase inhibitor of the present application, the agent capable of amplifying a nucleic acid of the present application, the dNTP of the present application and the lysing agent of the present application.

In the present application, said composition may comprise the tRNA of the present application, the RNase inhibitor of the present application, the reverse transcriptase of the present application, the dNTP of the present application and the lysing agent of the present application.

In the present application, said composition may comprise the tRNA of the present application, the RNase inhibitor of the present application, the reverse transcriptase of the present application, the dNTP of the present application and the Tween of the present application.

In the present application, said composition may comprise the tRNA of the present application, the inhibitor of RNase A, RNase B and/or RNase C of the present application, the reverse transcriptase of the present application, the dNTP of the present application and the Tween 20 of the present application.

In the present application, said composition may comprise about 0.01 μg/μl to about 1 μg/μl the tRNA of the present application, about 0.01 Unit/μl to about 1 Unit/μl the inhibitor of RNase A, RNase B and/or RNase C of the present application, about 0.1 Unit/μl to about 5 Unit/μl the reverse transcriptase of the present application, about 0.5 mM to about 5 mM each dNTP of the present application and about 0.01% (v/v) to about 1.5% (v/v) the Tween 20 of the present application.

In the present application, said composition may comprise at least about 0.05 μg/μl the tRNA of the present application, at least about 0.05 Unit/μl the inhibitor of RNase A, RNase B and/or RNase C of the present application, at least about 0.3 Unit/μl the reverse transcriptase of the present application, at least about 1.4 mM each dNTP of the present application and at least about 0.1% (v/v) Tween 20 of the present application.

### Primer and Isolated Oligonucleotide

In the present application, the composition further may comprise one or more primers specific for the target nucleic acid.

In another aspect, the present application provides a primer, which is capable of specifically amplifying a SARS-COV-2—associated nucleic acid.

In the present application, the one or more primers may be suitable for amplifying the target nucleic acid in an isothermal amplification.

In the present application, the isothermal amplification may comprise NASBA (nucleic acid sequence-based amplification), loop-mediated isothermal amplification (LAMP), recombinase polymerase amplification (RPA), and/or helicase-dependent amplification (HDA).

For example, the primer may not comprise 5 or more (for example, the primer may not comprise 5, 6, 7, 8, 9, 10 or more) consecutive identical single nucleotides. In the present application, the consecutive identical single nucleotide may comprise AAAA, TTTTT, CCCCCC or GGGGGGGGGGG. For example, the primer may not be able to comprise 5 or more consecutive identical single nucleotides.

For example, the primer may comprise at most one (for example, 0 or 1) fragment of 4 consecutive identical single nucleotide. For example, the primer may comprise one fragment of AAAA, TTTT, CCCC, or GGGG. For example, the primer may not comprise any 4 consecutive identical single nucleotides.

For example, the primer may comprise at most two (for example, 0, 1 or 2) fragments of 3 consecutive identical single nucleotide. For example, the primer may comprise AAA, TTT, CCC or GGG. For example, the primer may comprise at most two fragments selected from AAA, TTT, CCC and GGG. For example, the primer may not comprise any 3 consecutive identical single nucleotide.

In the present application, the primer may not comprise 4 or more (for example, 4, 5, 6, 7, 8, 9, 10 or more) consecutive dinucleotide repeats. In the present application, the consecutive dinucleotide repeats may comprise AA, TT, CC or GG. For example, the primer may comprise 0, 1, 2, or 3 the consecutive dinucleotide repeats AA, TT, CC or GG.

In the present application, the primer may not form any secondary structure with a ΔG lower than about −4 kcal/mol (For example, the primer may not form any secondary structure with a ΔG lower than about −5 kcal/mol, lower than about −6 kcal/mol, lower than about −7 kcal/mol, lower than about −8 kcal/mol, lower than about −9 kcal/mol, lower than about −10 kcal/mol, lower than about −20 kcal/mol, or even lower). For example, the primer may form with a relatively weak secondary structure. For example, the primer may not be able to form any secondary structure with a ΔG lower than about −4 kcal/mol.

In the present application, the ΔG may refer to Gibbs free energy change, which is the energy required to break the secondary structure. For example, larger negative values of the ΔG may indicate stable, undesirable hairpins that may adversely affect the amplification reaction.

For example, the primer FIP (Forward Inner Primer) and/or BIP (Backward Inner Primer) may not form any secondary structure with a ΔG lower than about −4 kcal/mol (For example, the primer may not form any secondary structure with a ΔG lower than about −5 kcal/mol, lower than about −6 kcal/mol, lower than about −7 kcal/mol, lower than about −8 kcal/mol, lower than about −9 kcal/mol, lower than about −10 kcal/mol, lower than about −20 kcal/mol, or even lower). For example, the primer F3 and/or B3, LF and/or LB may not form any secondary structure with a ΔG lower than about −3 kcal/mol (For example, the primer may not form any secondary structure with a ΔG lower than about −5 kcal/mol, lower than about −6 kcal/mol, lower than about −7 kcal/mol, lower than about −8 kcal/mol, lower than about −9 kcal/mol, lower than about −10 kcal/mol, lower than about −20 kcal/mol, or even lower).

In the present application, the primer may not comprise a 3′ end region having 6 or more nucleotides (for example, having 6, 7, 8, 9, 10 or more nucleotides) that are reverse complementary to a sequence within the same primer or within another primer of the more primers (for example, 1, 2, 3, 4, 5 or 6 primers). For example, the primer may not be able to comprise a 3′ end region having 6 or more nucleotides that are reverse complementary to a sequence within the same primer or within another primer of the more primers.

In the present application, the primer may not comprise a 3′ end region having 6 or more consecutive nucleotides (for example, having 6, 7, 8, 9, 10 or more nucleotides) that are reverse complementary to a sequence within the same primer or within another primer of the more primers (for example, 1, 2, 3, 4, 5 or 6 primers). For example, the primer may not be able to comprise a 3′ end region having 6 or more consecutive nucleotides that are reverse complementary to a sequence within the same primer or within another primer of the more primers.

In the present application, the target nucleic acid may be a pathogen-associated nucleic acid.

In the present application, the pathogen may be a virus.

In the present application, the target nucleic acid may be a virus RNA. For example, the virus RNA may comprise single-stranded RNA (ssRNA) and/or double-stranded RNA (dsRNA).

For example, the target nucleic acid may be a virus RNA originated from Coronaviridae. For example, the target nucleic acid may be a virus RNA originated from Sarbecovirus.

In the present application, the target nucleic acid may be a SARS-COV-2-associated nucleic acid, and wherein the primer may comprise a nucleic acid sequence that is 1) at least 98% homologous (for example, at least 99% homologous, at least 99.5% homologous or more) to the corresponding sequence of each known SARS-COV-2 strain, or 2) complementary to a nucleic acid sequence that is at least 98% (for example, at least 99% homologous, at least 99.5% homologous or more) homologous to the corresponding sequence of each known SARS-COV-2 strain.

For example, the target nucleic acid may be a target nucleic acid originated from strain H. Coronavirus 229E (NC_002645), a H. Coronavirus 229E (MN369046), a H. Coronavirus OC43 (KX344031.1), a H. Coronavirus OC43 (NC_006213), a H. Coronavirus HKUI (NC_006577), a H. Coronavirus HKUI (MK167038), a H. Coronavirus HKUI (MH940245), a H. Coronavirus NL63 (NC005831), a SARS Coronavirus (NC_004718), a MERS (NC_019843), a Adenovirus (AC_000017), a H. Metapneumovirus (NC_039199), a H. Parainfluenza 1 (AF457102), a H. Parainfluenza 4a (NC_021928), a H. Parainfluenza 4b (MN306032), a Influenza A (NC_026435), a Influenza B (NC_002205), a H. rhinovirus (NC_038311), a Chlamydia pneumoniae, a Legionella pneumophila, a Mycobacterium tuberculosis, a Streptococcus pneumoniae, a Streptococcus pyogenes, a Bordetella pertussis, a Mycoplasma pneumoniae, a Pneumocystis jirovecii, a Candida albicans, a Pseudomonas aeruginosa, a Staphylococcus epidermidis, or a Streptococcus salivarius.

For example, the target nucleic acid may be a N gene or the functional portion thereof, a M gene or the functional portion thereof, and/or an O gene or the functional portion thereof of the SARS-CoV-2. For example, the target nucleic acid may be a N gene or the functional portion thereof the SARS-COV-2. For example, the target nucleic acid may be other SARS-COV-2 variants such as B.1.1.7 (the UK strain) and B. 1.351 (the South Africa strain), influenza A, and influenza B, or respiratory syncytial virus (RSV).

In the present application, the target nucleic acid may be a SARS-COV-2-associated nucleic acid, and the one or more primers comprise primers 1-6, the primer 1 may comprise a nucleotide sequence as set forth in any of SEQ ID NO.3, SEQ ID NO.13, SEQ ID NO.33, SEQ ID NO.45, SEQ ID NO.63, SEQ ID NO.73, SEQ ID NO.83, SEQ ID NO.93, SEQ ID NO.106, SEQ ID NO.116, SEQ ID NO.126, SEQ ID NO.136, SEQ ID NO.146 and SEQ ID NO.156, the primer 2 may comprise a nucleotide sequence as set forth in any of SEQ ID NO.4, SEQ ID NO.14, SEQ ID NO.34, SEQ ID NO.46, SEQ ID NO.64, SEQ ID NO.74, SEQ ID NO.84, SEQ ID NO.94, SEQ ID NO.107, SEQ ID NO.117, SEQ ID NO.127, SEQ ID NO.137, SEQ ID NO.147 and SEQ ID NO.157, the primer 3 may comprise a nucleotide sequence as set forth in any of SEQ ID NO.1, SEQ ID NO.11, SEQ ID NO.31, SEQ ID NO.41, SEQ ID NO.43, SEQ ID NO.55, SEQ ID NO. 61, SEQ ID NO. 71, SEQ ID NO. 81, SEQ ID NO.91, SEQ ID NO.101, SEQ ID NO.104, SEQ ID NO.114, SEQ ID NO.124, SEQ ID NO.134, SEQ ID NO.144, SEQ ID NO.154 and SEQ ID NO.163, the primer 4 may comprise a nucleotide sequence as set forth in any of SEQ ID NO.2, SEQ ID NO.12, SEQ ID NO.32, SEQ ID NO.42, SEQ ID NO.44, SEQ ID NO.56, SEQ ID NO.62, SEQ ID NO.72, SEQ ID NO.82, SEQ ID NO.92, SEQ ID NO.102, SEQ ID NO.105, SEQ ID NO.115, SEQ ID NO.125, SEQ ID NO. 135, SEQ ID NO.145 and SEQ ID NO.155, the primer 5 may comprise a nucleotide sequence as set forth in any of SEQ ID NO.5, SEQ ID NO.15, SEQ ID NO.35, SEQ ID NO.47, SEQ ID NO.53, SEQ ID NO.55, SEQ ID NO.57, SEQ ID NO.59, SEQ ID NO.65, SEQ ID NO.75, SEQ ID NO.85, SEQ ID NO.95, SEQ ID NO.103, SEQ ID NO.108, SEQ ID NO.118, SEQ ID NO.128, SEQ ID NO.138, SEQ ID NO.148 and SEQ ID NO.158, and the primer 6 may comprise a nucleotide sequence as set forth in any of SEQ ID NO.6, SEQ ID NO.16, SEQ ID NO.36, SEQ ID NO.48, SEQ ID NO.54, SEQ ID NO.56, SEQ ID NO.58, SEQ ID NO.60, SEQ ID NO.66, SEQ ID NO.76, SEQ ID NO.86, SEQ ID NO.96, SEQ ID NO. 109, SEQ ID NO.119, SEQ ID NO.129, SEQ ID NO. 139 and SEQ ID NO.149.

For example, the set of primers may comprise: six primers each respectively having a sequence at least 90%, at least 95%, at least 96%, at least 97%, at least 98% or at least 99% identical to SEQ ID NO. 3 (FIP); SEQ ID NO. 4 (BIP); SEQ ID NO.1 (F3); SEQ ID NO. 2 (B3); SEQ ID NO. 5 (LF); and SEQ ID NO. 6 (LB).

For example, the set of primers may comprise: six primers each respectively having a sequence at least 90%, at least 95%, at least 96%, at least 97%, at least 98% or at least 99% identical to SEQ ID NO. 13 (FIP); SEQ ID NO. 14 (BIP); SEQ ID NO. 11 (F3); SEQ ID NO. 12 (B3); SEQ ID NO. 15 (LF); and SEQ ID NO. 16 (LB).

For example, the set of primers may comprise: six primers each respectively having a sequence at least 90%, at least 95%, at least 96%, at least 97%, at least 98% or at least 99% identical to SEQ ID NO. 33 (FIP); SEQ ID NO.34 (BIP); SEQ ID NO. 31 (F3); SEQ ID NO. 32 (B3); SEQ ID NO. 35 (LF); and SEQ ID NO. 36 (LB).

For example, the set of primers may comprise: six primers each respectively having a sequence at least 90%, at least 95%, at least 96%, at least 97%, at least 98% or at least 99% identical to SEQ ID NO. 33 (FIP); SEQ ID NO.34 (BIP); SEQ ID NO. 41 (F3); SEQ ID NO. 42 (B3); SEQ ID NO. 35 (LF); and SEQ ID NO. 36 (LB).

For example, the set of primers may comprise: six primers each respectively having a to SEQ ID NO. 45 (FIP); SEQ ID NO. 46 (BIP); SEQ ID NO. 43 (F3); SEQ ID NO. 44 (B3); SEQ ID NO. 47 (LF); and SEQ ID NO. 48 (LB).

For example, the set of primers may comprise: six primers each respectively having a sequence at least 90%, at least 95%, at least 96%, at least 97%, at least 98% or at least 99% identical to SEQ ID NO. 45 (FIP); SEQ ID NO. 46 (BIP); SEQ ID NO. 43 (F3); SEQ ID NO. 44 (B3); SEQ ID NO. 53 (LF); and SEQ ID NO. 54 (LB).

For example, the set of primers may comprise: six primers each respectively having a sequence at least 90%, at least 95%, at least 96%, at least 97%, at least 98% or at least 99% identical to SEQ ID NO. 45 (FIP); SEQ ID NO. 46 (BIP); SEQ ID NO. 43 (F3); SEQ ID NO. 44 (B3); SEQ ID NO. 55 (LF); and SEQ ID NO. 56 (LB).

For example, the set of primers may comprise: six primers each respectively having a sequence at least 90%, at least 95%, at least 96%, at least 97%, at least 98% or at least 99% identical to SEQ ID NO. 45 (FIP); SEQ ID NO. 46 (BIP); SEQ ID NO. 43 (F3); SEQ ID NO. 44 (B3); SEQ ID NO. 57 (LF); and SEQ ID NO. 58 (LB).

For example, the set of primers may comprise: six primers each respectively having a sequence at least 90%, at least 95%, at least 96%, at least 97%, at least 98% or at least 99% identical to SEQ ID NO. 45 (FIP); SEQ ID NO. 46 (BIP); SEQ ID NO. 43 (F3); SEQ ID NO. 44 (B3); SEQ ID NO.59 (LF); and SEQ ID NO. 60 (LB).

For example, the set of primers may comprise: six primers each respectively having a sequence at least 90%, at least 95%, at least 96%, at least 97%, at least 98% or at least 99% identical to SEQ ID NO. 63 (FIP); SEQ ID NO.64 (BIP); SEQ ID NO. 61 (F3); SEQ ID NO.62 (B3); SEQ ID NO. 65 (LF); and SEQ ID NO. 66 (LB).

For example, the set of primers may comprise: six primers each respectively having a sequence at least 90%, at least 95%, at least 96%, at least 97%, at least 98% or at least 99% identical to SEQ ID NO. 73 (FIP); SEQ ID NO. 74 (BIP); SEQ ID NO. 71 (F3); SEQ ID NO. 72 (B3); SEQ ID NO. 75 (LF); and SEQ ID NO. 76 (LB).

For example, the set of primers may comprise: six primers each respectively having a sequence at least 90%, at least 95%, at least 96%, at least 97%, at least 98% or at least 99% identical to SEQ ID NO. 83 (FIP); SEQ ID NO. 84 (BIP); SEQ ID NO. 81 (F3); SEQ ID NO. 82 (B3); SEQ ID NO. 85 (LF); and SEQ ID NO. 86 (LB).

For example, the set of primers may comprise: six primers each respectively having a to SEQ ID NO. 93 (FIP); SEQ ID NO. 94 (BIP); SEQ ID NO. 91 (F3); SEQ ID NO. 92 (B3); SEQ ID NO. 95 (LF); and SEQ ID NO. 96 (LB).

For example, the set of primers may comprise six primers each respectively having a sequence at least 90%, at least 95%, at least 96%, at least 97%, at least 98% or at least 99% identical to SEQ ID NO. 3 (FIP); SEQ ID NO. 4 (BIP); SEQ ID NO. 101 (F3); SEQ ID NO.102 (B3); SEQ ID NO. 103 (LF); and SEQ ID NO. 6 (LB).

For example, the set of primers may comprise six primers each respectively having a sequence at least 90%, at least 95%, at least 96%, at least 97%, at least 98% or at least 99% identical to SEQ ID NO. 106 (FIP); SEQ ID NO. 107 (BIP); SEQ ID NO. 104 (F3); SEQ ID NO. 105 (B3); SEQ ID NO. 108 (LF); and SEQ ID NO. 109 (LB).

For example, the set of primers may comprise six primers each respectively having a sequence at least 90%, at least 95%, at least 96%, at least 97%, at least 98% or at least 99% identical to SEQ ID NO. 116(FIP); SEQ ID NO. 117 (BIP); SEQ ID NO. 114 (F3); SEQ ID NO.115 (B3); SEQ ID NO.118 (LF); and SEQ ID NO. 119(LB).

For example, the set of primers may comprise six primers each respectively having a sequence at least 90%, at least 95%, at least 96%, at least 97%, at least 98% or at least 99% identical to SEQ ID NO. 126 (FIP); SEQ ID NO. 127 (BIP); SEQ ID NO.124 (F3); SEQ ID NO. 125(B3); SEQ ID NO. 128 (LF); and SEQ ID NO.129 (LB).

For example, the set of primers may comprise six primers each respectively having a sequence at least 90%, at least 95%, at least 96%, at least 97%, at least 98% or at least 99% identical to SEQ ID NO. 136 (FIP); SEQ ID NO. 137 (BIP); SEQ ID NO. 134 (F3); SEQ ID NO. 135 (B3); SEQ ID NO. 138(LF); and SEQ ID NO. 139 (LB).

For example, the set of primers may comprise six primers each respectively having a sequence at least 90%, at least 95%, at least 96%, at least 97%, at least 98% or at least 99% identical to SEQ ID NO. 146 (FIP); SEQ ID NO. 147 (BIP); SEQ ID NO. 144 (F3); SEQ ID NO. 145 (B3); SEQ ID NO. 148 (LF); and SEQ ID NO. 149 (LB).

For example, the set of primers may comprise six primers each respectively having a sequence at least 90%, at least 95%, at least 96%, at least 97%, at least 98% or at least 99% identical to SEQ ID NO. 156 (FIP); SEQ ID NO. 157 (BIP); SEQ ID NO. 154 (F3); SEQ ID NO. 155(B3); SEQ ID NO. 158 (LF); and SEQ ID NO.76 (LB).

For example, the set of primers may comprise six primers each respectively having a to SEQ ID NO. 156 (FIP); SEQ ID NO. 157 (BIP); SEQ ID NO. 163 (F3); SEQ ID NO. 155 (B3); SEQ ID NO. 158 (LF); and SEQ ID NO. 76 (LB).

For another example, the set of primers may comprise six primers each respectively having a sequence at least 90%, at least 95%, at least 96%, at least 97%, at least 98% or at least 99% identical to SEQ ID NO. 166 (FIP); SEQ ID NO. 167 (BIP); SEQ ID NO. 164 (F3); SEQ ID NO. 165 (B3); SEQ ID NO. 168 (LF); and SEQ ID NO. 169 (LB). And this set of primer may be used for amplifying human RNaseP gene.

For another example, the set of primers may comprise six primers each respectively having a sequence at least 90%, at least 95%, at least 96%, at least 97%, at least 98% or at least 99% identical to SEQ ID NO. 176 (FIP); SEQ ID NO. 177 (BIP); SEQ ID NO. 174 (F3); SEQ ID NO. 175 (B3); SEQ ID NO. 178 (LF); and SEQ ID NO. 179 (LB). And this set of primer may be used for amplifying human RNaseP gene.

In present application, the human RNaseP gene may be regarded as an internal control. For example, the RNase P Primer (RP) may be included in each run to detect the human RNaseP gene in the sample (for example, each saliva sample) of present application in order to monitor adequate RNA release from host cell present in the sample during the detection (for example, during the lysis step); also, it may be used to monitor reagent failure and the efficiency of the isothermal amplification (for example, the RT-LAMP) and end-point detection steps. The RP may also be used for monitoring inhibitors in the sample which may reduce the amplification efficiency.

In an example, the set of primers may comprise six primers each respectively having a sequence comprising or consisting of SEQ ID NO. 3 (FIP); SEQ ID NO. 4 (BIP); SEQ ID NO.1 (F3); SEQ ID NO. 2 (B3); SEQ ID NO. 5 (LF); and SEQ ID NO. 6 (LB).

In an example, the set of primers may comprise six primers each respectively having a sequence comprising or consisting of SEQ ID NO. 13 (FIP); SEQ ID NO. 14 (BIP); SEQ ID NO. 11 (F3); SEQ ID NO. 12 (B3); SEQ ID NO. 15 (LF); and SEQ ID NO. 16 (LB).

In an example, the set of primers may comprise six primers each respectively having a sequence comprising or consisting of SEQ ID NO. 33 (FIP); SEQ ID NO.34 (BIP); SEQ ID NO. 31 (F3); SEQ ID NO. 32 (B3); SEQ ID NO. 35 (LF); and SEQ ID NO. 36 (LB).

In an example, the set of primers may comprise six primers each respectively having a sequence comprising or consisting of SEQ ID NO. 33 (FIP); SEQ ID NO.34 (BIP); SEQ ID NO. 41 (F3); SEQ ID NO. 42 (B3); SEQ ID NO. 35 (LF); and SEQ ID NO. 36 (LB).

In an example, the set of primers may comprise six primers each respectively having a sequence comprising or consisting of SEQ ID NO. 45 (FIP); SEQ ID NO. 46 (BIP); SEQ ID NO. 43 (F3); SEQ ID NO. 44 (B3); SEQ ID NO. 47 (LF); and SEQ ID NO. 48 (LB).

In an example, the set of primers may comprise six primers each respectively having a sequence comprising or consisting of SEQ ID NO. 45 (FIP); SEQ ID NO. 46 (BIP); SEQ ID NO. 43 (F3); SEQ ID NO. 44 (B3); SEQ ID NO. 53 (LF); and SEQ ID NO. 54 (LB).

In an example, the set of primers may comprise six primers each respectively having a sequence comprising or consisting of SEQ ID NO. 45 (FIP); SEQ ID NO. 46 (BIP); SEQ ID NO. 43 (F3); SEQ ID NO. 44 (B3); SEQ ID NO. 55 (LF); and SEQ ID NO. 56 (LB).

In an example, the set of primers may comprise six primers each respectively having a sequence comprising or consisting of SEQ ID NO. 45 (FIP); SEQ ID NO. 46 (BIP); SEQ ID NO. 43 (F3); SEQ ID NO. 44 (B3); SEQ ID NO. 57 (LF); and SEQ ID NO. 58 (LB).

In an example, the set of primers may comprise six primers each respectively having a sequence comprising or consisting of SEQ ID NO. 45 (FIP); SEQ ID NO. 46 (BIP); SEQ ID NO. 43 (F3); SEQ ID NO. 44 (B3); SEQ ID NO.59 (LF); and SEQ ID NO. 60 (LB).

In an example, the set of primers may comprise six primers each respectively having a sequence comprising or consisting of SEQ ID NO. 63 (FIP); SEQ ID NO.64 (BIP); SEQ ID NO. 61 (F3); SEQ ID NO.62 (B3); SEQ ID NO. 65 (LF); and SEQ ID NO. 66 (LB).

In an example, the set of primers may comprise six primers each respectively having a sequence comprising or consisting of SEQ ID NO. 73 (FIP); SEQ ID NO. 74 (BIP); SEQ ID NO. 71 (F3); SEQ ID NO. 72 (B3); SEQ ID NO. 75 (LF); and SEQ ID NO. 76 (LB).

In an example, the set of primers may comprise six primers each respectively having a sequence comprising or consisting of SEQ ID NO. 83 (FIP); SEQ ID NO. 84 (BIP); SEQ ID NO. 81 (F3); SEQ ID NO. 82 (B3); SEQ ID NO. 85 (LF); and SEQ ID NO. 86 (LB).

In an example, the set of primers may comprise six primers each respectively having a sequence comprising or consisting of SEQ ID NO. 93 (FIP); SEQ ID NO. 94 (BIP); SEQ ID NO. 91 (F3); SEQ ID NO. 92 (B3); SEQ ID NO. 95 (LF); and SEQ ID NO. 96 (LB).

In an example, the set of primers may comprise six primers each respectively having a sequence comprising or consisting of SEQ ID NO. 3 (FIP); SEQ ID NO. 4 (BIP); SEQ ID NO. 101 (F3); SEQ ID NO.102 (B3); SEQ ID NO. 103 (LF); and SEQ ID NO. 6 (LB).

In an example, the set of primers may comprise six primers each respectively having a sequence comprising or consisting of SEQ ID NO. 106 (FIP); SEQ ID NO. 107 (BIP); SEQ ID NO. 104 (F3); SEQ ID NO. 105 (B3); SEQ ID NO. 108 (LF); and SEQ ID NO. 109 (LB).

In an example, the set of primers may comprise six primers each respectively having a sequence comprising or consisting of SEQ ID NO. 116(FIP); SEQ ID NO. 117 (BIP); SEQ ID NO. 114 (F3); SEQ ID NO.115 (B3); SEQ ID NO.118 (LF); and SEQ ID NO. 119(LB).

In an example, the set of primers may comprise six primers each respectively having a sequence comprising or consisting of SEQ ID NO. 126 (FIP); SEQ ID NO. 127 (BIP); SEQ ID NO.124 (F3); SEQ ID NO. 125(B3); SEQ ID NO. 128 (LF); and SEQ ID NO.129 (LB).

In an example, the set of primers may comprise six primers each respectively having a sequence comprising or consisting of SEQ ID NO. 136 (FIP); SEQ ID NO. 137 (BIP); SEQ ID NO. 134 (F3); SEQ ID NO. 135 (B3); SEQ ID NO. 138(LF); and SEQ ID NO. 139 (LB).

In an example, the set of primers may comprise six primers each respectively having a sequence comprising or consisting of SEQ ID NO. 146 (FIP); SEQ ID NO. 147 (BIP); SEQ ID NO. 144 (F3); SEQ ID NO. 145 (B3); SEQ ID NO. 148 (LF); and SEQ ID NO. 149 (LB).

In an example, the set of primers may comprise six primers each respectively having a sequence comprising or consisting of SEQ ID NO. 156 (FIP); SEQ ID NO. 157 (BIP); SEQ ID NO. 154 (F3); SEQ ID NO. 155(B3); SEQ ID NO. 158 (LF); and SEQ ID NO.76 (LB).

In an example, the set of primers may comprise six primers each respectively having a sequence comprising or consisting of SEQ ID NO. 156 (FIP); SEQ ID NO. 157 (BIP); SEQ ID NO. 163 (F3); SEQ ID NO. 155 (B3); SEQ ID NO. 158 (LF); and SEQ ID NO. 76 (LB).

In another example, the set of primers may comprise six primers each respectively having a sequence comprising or consisting of SEQ ID NO. 166 (FIP); SEQ ID NO. 167 (BIP); SEQ ID NO. 164 (F3); SEQ ID NO. 165 (B3); SEQ ID NO. 168 (LF); and SEQ ID NO. 169 (LB).

In another example, the set of primers may comprise six primers each respectively having a sequence comprising or consisting of SEQ ID NO. 176 (FIP); SEQ ID NO. 177 (BIP); SEQ ID NO. 174 (F3); SEQ ID NO. 175 (B3); SEQ ID NO. 178 (LF); and SEQ ID NO. 179 (LB).

In present application, the primer FIP may comprise or be consisted of two fragments F1C and F2.

For example, SEQ ID NO. 3 (FIP) may comprise or be consisted of two fragments SEQ ID NO. 7 (F1C) and SEQ ID NO. 8 (F2).

For example, SEQ ID NO. 13 (FIP) may comprise or be consisted of two fragments SEQ ID NO. 17 (F1C) and SEQ ID NO. 18 (F2).

For example, SEQ ID NO. 33 (FIP) may comprise or be consisted of two fragments SEQ ID NO. 37 (F1C) and SEQ ID NO. 38 (F2).

For example, SEQ ID NO. 45 (FIP) may comprise or be consisted of two fragments SEQ ID NO. 49 (F1C) and SEQ ID NO.50 (F2).

For example, SEQ ID NO. 63 (FIP) may comprise or be consisted of two fragments SEQ ID NO. 67 (F1C) and SEQ ID NO. 68 (F2).

For example, SEQ ID NO. 73 (FIP) may comprise or be consisted of two fragments SEQ ID NO. 77 (F1C) and SEQ ID NO. 78 (F2).

For example, SEQ ID NO. 83 (FIP) may comprise or be consisted of two fragments SEQ ID NO. 87 (F1C) and SEQ ID NO. 88 (F2).

For example, SEQ ID NO. 93 (FIP) may comprise or be consisted of two fragments SEQ ID NO. 97 (F1C) and SEQ ID NO. 98 (F2).

For example, SEQ ID NO. 3 (FIP) may comprise or be consisted of two fragments SEQ ID NO. 7 (F1C) and SEQ ID NO. 8 (F2).

For example, SEQ ID NO.106 (FIP) may comprise or be consisted of two fragments SEQ ID NO. 110 (F1C) and SEQ ID NO. 111 (F2).

For example, SEQ ID NO. 116 (FIP) may comprise or be consisted of two fragments SEQ ID NO. 120 (F1C) and SEQ ID NO. 121 (F2).

For example, SEQ ID NO. 126 (FIP) may comprise or be consisted of two fragments SEQ ID NO. 130 (F1C) and SEQ ID NO. 131 (F2).

For example, SEQ ID NO. 136 (FIP) may comprise or be consisted of two fragments SEQ ID NO. 140 (F1C) and SEQ ID NO. 141 (F2).

For example, SEQ ID NO. 146 (FIP) may comprise or be consisted of two fragments SEQ ID NO. 150 (F1C) and SEQ ID NO. 151 (F2).

For example, SEQ ID NO. 156 (FIP) may comprise or be consisted of two fragments SEQ ID NO. 159 (F1C) and SEQ ID NO. 160 (F2).

For example, SEQ ID NO. 166 (FIP) may comprise or be consisted of two fragments SEQ ID NO. 170 (F1C) and SEQ ID NO. 171 (F2).

For example, SEQ ID NO. 176 (FIP) may comprise or be consisted of two fragments SEQ ID NO. 180 (F1C) and SEQ ID NO. 181 (F2).

In present application, the primer BIP may comprise or be consisted of two fragments B1C and B2.

For example, SEQ ID NO. 4 (BIP) may comprise or be consisted of two fragments SEQ ID NO. 9 (B1C) and SEQ ID NO. 10 (B2).

For example, SEQ ID NO. 14 (BIP) may comprise or be consisted of two fragments SEQ ID NO. 19 (B1C) and SEQ ID NO. 20 (B2).

For example, SEQ ID NO. 34 (BIP) may comprise or be consisted of two fragments SEQ ID NO. 39 (B1C) and SEQ ID NO. 40 (B2).

For example, SEQ ID NO. 46 (BIP) may comprise or be consisted of two fragments SEQ ID NO. 51 (B1C) and SEQ ID NO. 52 (B2).

For example, SEQ ID NO. 64 (BIP) may comprise or be consisted of two fragments SEQ ID NO. 69 (B1C) and SEQ ID NO. 70 (B2).

For example, SEQ ID NO. 74 (BIP) may comprise or be consisted of two fragments SEQ ID NO. 79 (B1C) and SEQ ID NO. 80 (B2).

For example, SEQ ID NO. 84 (BIP) may comprise or be consisted of two fragments SEQ ID NO. 89 (B1C) and SEQ ID NO. 90 (B2).

For example, SEQ ID NO. 94 (BIP) may comprise or be consisted of two fragments SEQ ID NO. 99 (B1C) and SEQ ID NO. 100 (B2).

For example, SEQ ID NO. 107 (BIP) may comprise or be consisted of two fragments SEQ ID NO. 112 (B1C) and SEQ ID NO. 113 (B2).

For example, SEQ ID NO. 117 (BIP) may comprise or be consisted of two fragments SEQ ID NO. 122 (B1C) and SEQ ID NO. 123 (B2).

For example, SEQ ID NO. 127 (BIP) may comprise or be consisted of two fragments SEQ ID NO. 132 (B1C) and SEQ ID NO. 133 (B2).

For example, SEQ ID NO. 137 (BIP) may comprise or be consisted of two fragments SEQ ID NO. 142 (B1C) and SEQ ID NO. 143 (B2).

For example, SEQ ID NO. 147 (BIP) may comprise or be consisted of two fragments SEQ ID NO. 152 (B1C) and SEQ ID NO. 153 (B2).

For example, SEQ ID NO. 157 (BIP) may comprise or be consisted of two fragments SEQ ID NO. 161 (B1C) and SEQ ID NO. 162 (B2).

For example, SEQ ID NO. 167 (BIP) may comprise or be consisted of two fragments SEQ ID NO. 172 (B1C) and SEQ ID NO. 173 (B2).

For example, SEQ ID NO. 177 (BIP) may comprise or be consisted of two fragments SEQ ID NO. 182 (B1C) and SEQ ID NO. 183 (B2).

In the present application, the following set of primers may be regarded as a comparative primer for detecting the SARS-COV-2: six primers each having a sequence as set forth in SEQ ID NO. 23 (FIP); SEQ ID NO. 24 (BIP); SEQ ID NO. 21 (F3); SEQ ID NO. 22 (B3); SEQ ID NO. 25 (LF); and SEQ ID NO. 26 (LB).

In the present application, the target nucleic acid may be a SARS-COV-2-associated nucleic acid, and wherein the last 5 nucleotides from the extension start position of each primer are 100% homologous to the corresponding sequence of each known SARS-COV-2 strain, or 2) are complementary to a nucleic acid sequence that is 100% homologous to the corresponding sequence of each known SARS-COV-2 strain.

For example, the last 5 nucleotides from the 3′ end of primer F2, B2, F3, B3, LF and/or LB may be 100% homologous to the corresponding sequence of each known SARS-COV-2 strain. For example, the last 5 nucleotides from the 5′ end of primer Flc and/or Blc may be 100% homologous to the corresponding sequence of each known SARS-COV-2 strain.

In the present application, the primers may be between 10 and 60 nucleotides in length (for example 15-50, 20-50, 30-60, or 25-40 nucleotides in length). For example, the primers may be 10, 11, 12, 13, 14, 15, 16, 17, 18, 19, 20, 21, 22, 23, 24, 25, 26, 27, 28 29, 30, 31, 32, 32, 34, 35, 36, 37, 38, 39, 40, 41, 42, 43, 44, 45, 46, 47, 48, 49, 50, 51, 52, 53, 54, 55, 56, 57, 58, 59, or 60 nucleotides in length. For example, the primers may be at least 10, 15, 20, 25, 30, 35, 40, 45, 50, 55, or 60 nucleotides in length. For example, the primers may be no more than 20, 25, 30, 35, 40, 45, 50, 55, or 60 nucleotides in length.

In the present application, at least one of the primers may comprise a detectable label, such as a fluorophore, radiolabel, hapten (such as biotin), or chromogen. For example, a detectable label may be able to be attached (e.g., covalently or non-covalently attached) to an oligonucleotide. The attachment may be to any portion of the oligonucleotide, comprising a base, sugar, phosphate backbone, or 5′ or 3′ end of the oligonucleotide. The label may be directly attached to the oligonucleotide or indirectly attached, for example through a linker molecule. For example, the primer of the present application may comprise a fluorophore at the 5 Or 3′ end. In some examples, the fluorophore is HEX, FAM, TET, fluorescein, fluorescein isothiocyanate (FITC), or QFITC (XRITC).

In the present application, the primer sequences may be varied slightly by moving the primer a few nucleotides upstream or downstream from the nucleotide positions that they hybridize to on the target nucleic molecule acid, provided that the probe and/or primer may still be able to specific for the target nucleic acid sequence. For example, variations of the primers of the present application may be made by “sliding” the probes or primers a few nucleotides 5′ or 3′ from their positions, and such variations may still be specific for the respective target nucleic acid sequence.

In the present application, the primer sequences may be slightly longer or shorter than the nucleotide sequences shown in present application, as long as such deletions or additions permit amplification and/or detection of the desired target nucleic acid molecule. For example, a primer may comprise a few nucleotide deletions or additions at the 5′- or 3′-end of the primers of present application, such as addition or deletion of 1, 2, 3, 4, 5, or 6 nucleotides from the 5′- or 3′-end, or combinations thereof (such as a deletion from one end and an addition to the other end). In such examples, the number of nucleotides may change.

For example, the primer of present application may be able to degenerate at one or more positions (such as 1, 2, 3, 4, 5, or more positions), for example, a primer that may comprise a mixture of nucleotides (such as 2, 3, or 4 nucleotides) at a specified position in the primer. For example, the primer of the present application may comprise one or more synthetic (e.g., non-naturally occurring) bases or alternative bases (such as inosine). For another example, the primer of present application may comprise one or more modified nucleotides or nucleic acid analogues, such as one or more locked nucleic acids (see, e.g., U.S. Pat. No. 6,794,499), an altered sugar moiety, an inter-sugar linkage, a non-naturally occurring nucleotide linkage, a phosphorothioate oligodeoxynucleotide, a peptide nucleic acid (PNA), or one or more superbases (Nanogen, Inc., Bothell, WA).

In the present application, each of the one or more primers may be present in a concentration of about 0.05 μM to about 10 μM. For example, each of the one or more primers may be present in a concentration of about 1 μM to about 2 μM, about 1.6 μM to about 2 μM, about 0.1 μM to about 0.5 μM, about 0.2 μM to about 0.5 μM, about 0.3 μM to about 0.8 μM, about 0.4 μM to about 0.8 μM, about 0.1 μM to about 10 μM, about 2 μM to about 4 μM, about 3.2 μM to about 4 μM, about 0.2 μM to about 1.0 μM, about 0.4 μM to about 1.0 μM, about 0.6 μM to about 1.6 μM or about 0.8 μM to about 1.6 μM. For example, each of the one or more primers may be present in a concentration of at least about 0.2 μM, at least about 0.4 μM, at least about 0.6 μM, at least about 1.0 μM, at least about 1.6 μM, at least about 2 μM, at least about 3.2 μM, at least about 4 μM, or at least about 10 μM.

In the present application, each of the one or more primers may be present in a concentration of at least about 0.1 μM, at least about 0.2 μM, at least about 0.3 μM, at least about 0.4 μM, at least about 0.5 μM, at least about 0.8 μM, at least about 1 μM, at least about 1.6 μM, at least about 2 μM, at least about 5 μM, at least about 0.2 μM, at least about 0.4 μM, at least about 0.6 μM, at least about 1.0 μM, at least about 1.6 μM, at least about 2 μM, at least about 3.2 μM, at least about 4 μM, or at least about 10 μM.

For example, in the composition of present application, each of the one or more primers may be present in a concentration of about 0.05 μM to about 5 μM. For example, each of the one or more primers may be present in a concentration of about 1 μM to about 2 μM, about 1.6 μM to about 2 μM, about 0.1 μM to about 0.5 μM, about 0.2 μM to about 0.5 μM, about 0.3 μM to about 0.8 μM or about 0.4 μM to about 0.8 μM. For example, each of the one or more primers may be present in a concentration of at least about 0.1 μM, at least about 0.2 μM, at least about 0.3 μM, at least about 0.4 μM, at least about 0.5 μM, at least about 0.8 μM, at least about 1 μM, at least about 1.6 μM, at least about 2 μM, or at least about 5 μM.

For example, in the stocking solution of present application, each of the one or more primers may be present in a concentration of about 0.1 μM to about 10 μM. For example, each of the one or more primers may be present in a concentration of about 2 μM to about 4 μM, about 3.2 μM to about 4 μM, about 0.2 μM to about 1.0 μM, about 0.4 μM to about 1.0 μM, about 0.6 μM to about 1.6 μM or about 0.8 μM to about 1.6 μM. For example, each of the one or more primers may be present in a concentration of at least about 0.2 μM, at least about 0.4 μM, at least about 0.6 μM, at least about 1.0 μM, at least about 1.6 μM, at least about 2 μM, at least about 3.2 μM, at least about 4 μM, or at least about 10 μM.

For example, the primer 1 (for example, primer FIP) may be present in a concentration of about 1 μM to about 2 μM, the primer 2 (for example, primer BIP) is present in a concentration of about 1 μM to about 2 μM, the primer 3 (for example, primer F3) is present in a concentration of about 0.1 μM to about 0.5 μM, the primer 4 (for example, primer B3) is present in a concentration of about 0.1 μM to about 0.5 μM, the primer 5 (for example, primer LF) is present in a concentration of about 0.3 μM to about 0.8 μM, and the primer 6 (for example, primer LB) is present in a concentration of about 0.3 μM to about 0.8 μM.

For example, the primer 1 may be present in a concentration of at least about 1.6 μM.

For example, the primer 2 may be present in a concentration of at least about 1.6 μM.

For example, the primer 3 may be present in a concentration of at least about 0.2 μM.

For example, the primer 4 may be present in a concentration of at least about 0.2 μM.

For example, the primer 5 may be present in a concentration of at least about 0.4 μM.

For example, the primer 6 may be present in a concentration of at least about 0.4 μM.

For example, the primer 1 may be present in a concentration of at least about 1.6 μM; the primer 2 may be present in a concentration of at least about 1.6 μM; the primer 3 may be present in a concentration of at least about 0.2 μM; the primer 4 may be present in a concentration of at least about 0.2 μM; the primer 5 may be present in a concentration of at least about 0.4 μM and the primer 6 may be present in a concentration of at least about 0.4 μM.

In another aspect, the present application provides an isolated oligonucleotide, comprising a sequence as set forth in any of SEQ ID Nos. 1-20, 31-183.

For example, the isolated oligonucleotide may be capable of specifically binding to the virus RNA. For example, the virus RNA may comprise single-stranded RNA (ssRNA) and/or double-stranded RNA (dsRNA). For example, the isolated oligonucleotide may be capable of specifically binding to a virus RNA originated from Coronaviridae. For example, the isolated oligonucleotide may be capable of specifically binding to a virus RNA originated from Sarbecovirus. For example, the isolated oligonucleotide may be capable of specifically binding to a SARS-COV-2-associated nucleic acid.

In the present application, the isolated oligonucleotide may be capable of amplifying the target nucleic acid, for example, may be capable of amplifying the SARS-COV-2-associated nucleic acid. For example, the isolated oligonucleotide may be capable of amplifying a S protein or the functional portion thereof of the SARS-COV-2.

In the present application, the composition may comprise the nuclease inhibitor of the present application, the decoy oligonucleotide of the present application, the reverse transcriptase of the present application, the isothermal amplification buffer of the present application, the lysing agent of the present application, the dNTP of the present application and the primers of the present application.

In the present application, said composition may comprise about 0.01 μg/μl to about 1 μg/μl the tRNA of the present application, about 0.01 Unit/μl to about 1 Unit/μl the inhibitor of RNase A, RNase B and/or RNase C of the present application, about 0.1 Unit/μl to about 5 Unit/μl the reverse transcriptase of the present application, about 0.5 mM to about 5 mM each dNTP of the present application, about 0.01% (v/v) to about 1.5% (v/v) the Tween 20 of the present application and the primers (about 0.05 mM to about 5 mM).

In the present application, said composition may comprise about 0.05 μg/μl the tRNA of the present application, about 0.05 Unit/μl the inhibitor of RNase A, RNase B and/or RNase C of the present application, about 0.3 Unit/μl the reverse transcriptase of the present application, about 1.4 mM each dNTP of the present application, about 0.1% (v/v) Tween 20 of the present application and the primers (about 0.05 mM to about 5 mM).

For example, the composition may comprise the tRNA of the present application, the inhibitor of RNase A, RNase B and/or RNase C of the present application, the reverse transcriptase, the isothermal amplification buffer, Tween 20, the dNTP and the primers of the present application. For example, the composition may comprise the RNase inhibitor (about 0.03-about 0.75 Unit/μl), the RNA (about 0.01-about 0.25 μg/μl), the reverse transcriptase (about 0.1-about 0.3 Unit/μl), the isothermal amplification buffer, Tween 20 (about 0.01% (v/v) to about 1.5% (v/v)), the dNTP (about 0.5 mM to about 5 mM) and the primers (about 0.05 mM to about 5 mM).

For example, the composition may comprise the RNase inhibitor (about at least about 0.15 Unit/μl), the tRNA (about at least about 0.05 μg/μl), the reverse transcriptase (about at least about 0.3 Unit/μl), the isothermal amplification buffer, Tween 20 (about at least about 0.1 (v/v)), the dNTP (about at least about 1.4 mM) and the primers (about at least about 1.6 μM for primer 1 and primer 2; about at least about 0.2 μM for primer 3 and primer 4; about at least about 0.4 μM for primer 5 and primer 6).

### Sample

In the present application, the sample may comprise any material that contains, or potentially contains, which could be infected or contaminated by the presence of a pathogenic microorganism.

In the present application, the sample may be a biological sample.

In the present application, the sample may be selected from the group consisting of: nasopharyngeal swab, blood, serum, urine, saliva, tissue, cell, organ, a fraction or portion thereof, and any combination of the foregoing.

In other cases, the sample may also be a non-biological sample. For example, the sample may comprise plastic and packaging materials, paper, clothing fibers, and metal surfaces.

In the present application, the sample may be a crude sample derived directly from a subject without being processed. For example, the target nucleic acid in the sample may have not been subjected to amplification and/or purification.

In the present application, the composition may further comprise an electroactive indicator. The electroactive indicator may be able to indicate the existence of the target nucleic acid in the sample using the composition of the present application.

For example, the electroactive indicator may generate an electric signal when the target nucleic acid in the sample is contacted with the composition of the present application. For example, the electric indicator may comprise a redox current.

In the present application, the electroactive indicator may comprise methylene blue (MB). In the present application, before the target nucleic acid is contacted with the composition of the present application (for example, before the target nucleic acid is amplified with the composition of the present application), the MB may be free in solution and yields a high redox current at an electrode. During amplifying the target nucleic acid, MB may be able to intercalate into the newly formed double-stranded target nucleic acid, and the current may be decreased as a result.

In the present application, the electroactive indicator may measure the redox current in real-time. And the electroactive indicator may yield a current trace as a function of reaction time. Thus, the electroactive indicator may detect the target nucleic acid fast.

For example, the composition of the present application may be able to detect the target nucleic acid (for example, the SARS-COV-2) within 20 mins.

### Kit, System and Method

In another aspect, the present application provides a kit for detecting a target nucleic acid in a sample, comprising the composition of the present application, the isolated oligonucleotide of the present application, the primers of the present application, and/or the primer set of the present application.

In another aspect, the present application provides a use of the composition of the present application, the isolated oligonucleotide of the present application, the primers of the present application, and/or the primer set of the present application in the manufacture of an agent for detecting a target nucleic acid in a sample.

In the present application, the sample may be selected from the group consisting of: nasopharyngeal swab, blood, serum, urine, saliva, tissue, cell, organ, a fraction or portion thereof, and any combination of the foregoing.

In the present application, the sample may be a crude sample derived directly from a subject without being the processed.

In the present application, the target nucleic acid may be a pathogen-associated nucleic acid.

In the present application, the target nucleic acid may be a SARS-COV-2-associated nucleic acid.

In another aspect, the present application provides a system for detecting a target nucleic acid in a sample, comprising a sample analyzing module and a display module, wherein the sample analyzing module and the display module are informationally connected, the sample analyzing module comprise the composition of the present application, the isolated oligonucleotide of the present application, the primers of the present application, and/or the primer set of the present application, and the display module is configured to indicate a presence and/or amount of the target nucleic acid in the sample based on an information generated by the sample analyzing module.

In the present application, the signal may comprise an electric signal and/or a fluorescent signal. For example, the signal may comprise a current.

In another aspect, the present application provides a method for detecting a target nucleic acid in a sample, comprising administering the composition of the present application, the isolated oligonucleotide of the present application, the primers of the present application, and/or the primer set of the present application.

In another aspect, the present application provides a method, comprising: contacting a sample suspected to contain a target nucleic acid with the composition of the present application to obtain a one pot reaction mixture.

In the present application, the sample may be selected from the group consisting of: nasopharyngeal swab, blood, serum, urine, saliva, tissue, cell, organ, a fraction or portion thereof, and any combination of the foregoing.

In the present application, in the one pot reaction mixture, the nuclease inhibitor may present in a concentration of about 0.01Unit/μl to about 1 Unit/μl.

In the present application, in the one pot reaction mixture, the decoy oligonucleotide may present in a concentration of about 0.01 μg/μl to about 1 μg/μl.

In the present application, in the one pot reaction mixture, the agent capable of amplifying a nucleic acid may present in a concentration of about 0.1 Unit/μl to about 5 Unit/μl.

In the present application, in the one pot reaction mixture, the reverse transcriptase may present in a concentration of about 0.1 Unit/μl to about 5 Unit/μl.

In the present application, in the one pot reaction mixture, the lysing agent may present in a concentration of about 0.01% (v/v) to about 1.5% (v/v).

In the present application, in the one pot reaction mixture, each dNTPs may present in a concentration of about 0.5 mM to about 5 mM.

In the present application, in the one pot reaction mixture, the primers may present in a concentration of about 0.5 μM to about 5 μM.

In the present application, the sample may be a crude sample derived directly from a subject without being processed.

In the present application, the sample may have not been subjected to nucleic acid purification, cell lysis, and/or nucleic acid amplification.

In the present application, the method may further comprise: subjecting the one pot reaction mixture to nucleic acid amplification.

In the present application, the nucleic acid amplification may comprise an isothermal nucleic acid amplification reaction.

In the present application, the nucleic acid amplification may comprise NASBA, LAMP, and/or RPA. For example, the nucleic acid amplification may comprise LAMP.

For example, the present application may provide a method, comprising: contacting a sample suspected to contain a target nucleic acid with the composition of the present application to obtain a one pot reaction mixture, and subjecting the one pot reaction mixture to nucleic acid amplification.

For example, the present application may provide a method, comprising: contacting the crude sample of the present application suspected to contain a target nucleic acid with the tRNA of the present application, the nuclease inhibitor of the present application, the agent capable of amplifying a nucleic acid of the present application, the dNTP of the present application and the lysing agent of the present application to obtain a one pot reaction mixture, and subjecting the one pot reaction mixture to the isothermal nucleic acid amplification reaction.

For example, the present application may provide a method, comprising: contacting the crude sample of the present application suspected to contain a target nucleic acid with tRNA of the present application, the RNase inhibitor of the present application, the agent capable of amplifying a nucleic acid of the present application, the dNTP of the present application and the lysing agent of the present application to obtain a one pot reaction mixture, and subjecting the one pot reaction mixture to the isothermal nucleic acid amplification reaction.

For example, the present application may provide a method, comprising: contacting the crude sample of the present application suspected to contain a target nucleic acid with tRNA of the present application, the RNase inhibitor of the present application, the reverse transcriptase of the present application, the dNTP of the present application and the lysing agent of the present application to obtain a one pot reaction mixture, and subjecting the one pot reaction mixture to the isothermal nucleic acid amplification reaction.

For example, the present application may provide a method, comprising: contacting the crude sample of the present application suspected to contain a target nucleic acid with the tRNA of the present application, the RNase inhibitor of the present application, the reverse transcriptase of the present application, the dNTP of the present application and the Tween to obtain a one pot reaction mixture, and subjecting the one pot reaction mixture to the isothermal nucleic acid amplification reaction.

For example, the present application may provide a method, comprising: contacting the crude sample of the present application suspected to contain a target nucleic acid with the tRNA of the present application, the inhibitor of RNase A, RNase B and/or RNase C of the present application, the reverse transcriptase of the present application, the dNTP of the present application and the Tween 20 of the present application to obtain a one pot reaction mixture, and subjecting the one pot reaction mixture to the LAMP reaction.

In the present application, the nucleic acid amplification may be conducted at a single temperature of about 55° C. to about 70° C. (for example, may be conducted at a single temperature of about 55° C. to about 65° C., about 55° C. to about 60° C., about 56° C. to about 70° C., about 57° C. to about 70° C., about 58° C. to about 70° C., about 59° C. to about 70° C., or about 60° C. to about 70° C.). For example, the nucleic acid amplification may be conducted at a single temperature of about 55° C.

In the present application, before and/or during the nucleic acid amplification, an electric potential may be applied to the reaction mixture, and a presence and/or an amount of an amplification product may be indicated by a change of an electric signal and/or a fluorescent signal generated during and/or after the nucleic acid amplification.

In the present application, the electric signal may be a redox current, and a decrease of the redox current indicates a presence and/or increase of the amplification product.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); nt, nucleotide(s); i.m., intramuscular(ly); i.p., intraperitoneal(ly); s.c., subcutaneous(ly); and the like.

### Example 1

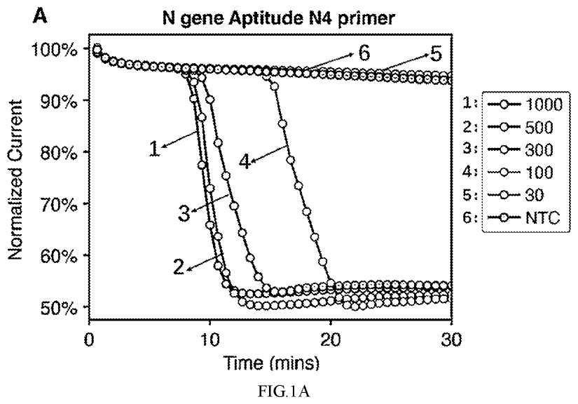

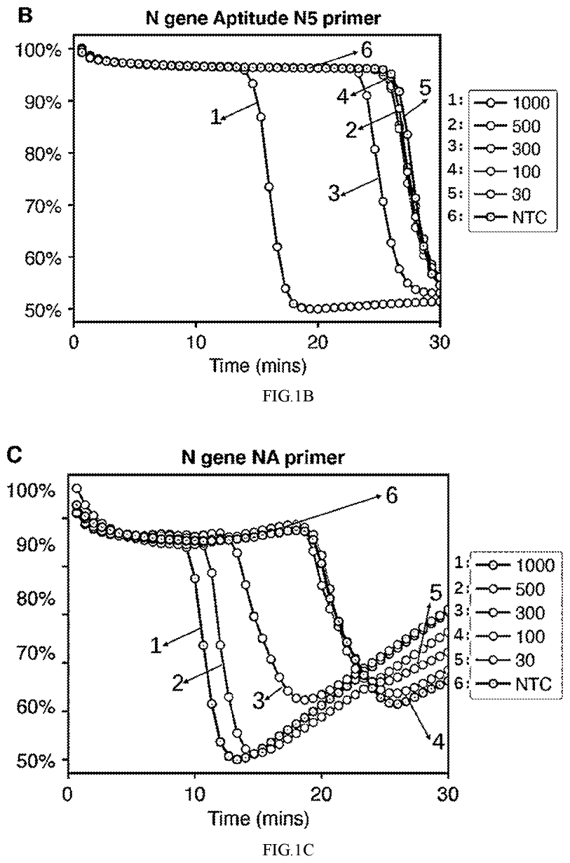

24 sets of LAMP primers targeting different regions of the SARS-COV-2 genome were tested. After thorough testing, a novel LAMP primer set for amplifying N gene of the SARS-COV-2, named Aptitude N4 primer set was identified, with which ˜100 copies of the SARS-COV-2 RNA (Twist Bioscience, 102024) per sample can be detected and yield maximum electrochemical current signal reduction (˜50%) within 30 minutes (FIG. 1A). In FIG. 1A, the result was measured by real-time electrochemical current across a range of concentrations (0 to 100 copies of SARS-COV-2 viral RNA per reaction) of SARS-COV-2 genomic standards. In FIG. 1A, the lines 1-5 are corresponding to the result of the different concentrations of SARS-COV-2 viral RNA per reaction, and the line 6 is corresponding to that of the NTC.

Moreover, the N4 primer set showed no non-specific amplification and negligible signal reduction for the no template control (NTC), showing superior specificity than the established LAMP primer set 46 (NA) and another set of in-house primer (N5) for the N gene (FIG. 1B-C).

The N4 primer comprises the following sequences: SEQ ID NO. 3 (N4-FIP); SEQ ID NO. 4 (N4-BIP); SEQ ID NO.1 (N4-F3); SEQ ID NO. 2 (N4-B3); SEQ ID NO. 5 (N4-LF); and SEQ ID NO. 6 (N4-LB).

The N5 primer comprises the following sequences: SEQ ID NO. 13 (N5-FIP); SEQ ID NO. 14 (N5-BIP); SEQ ID NO. 11 (N5-F3); SEQ ID NO. 12 (N5-B3); SEQ ID NO. 15 (N5-LF); and SEQ ID NO. 16 (N5-LB).

The NA primer comprises the following sequences: SEQ ID NO. 23 (NA-FIP); SEQ ID NO. 24 (NA-BIP); SEQ ID NO. 21 (NA-F3); SEQ ID NO.22 (NA-B3); SEQ ID NO. 25 (NA-LF); and SEQ ID NO. 26 (NA-LB).

### Example 2

The system of the present application provides a simple sample-to-answer POCT-format with saliva and swab samples.

The current system starts from saliva or nasopharyngeal swab collection into a barcoded sample collection tube containing the composition, allowing direct testing from unprocessed patient samples and making it readily deployable to home and public setting and used by lay-persons. It was validated by successfully detecting 100 copies of heat-inactivated SARS-COV-2 virus (ATCC, VR-1986HK) spiked into pooled healthy saliva or nasopharyngeal (NP) swab samples. The non-processed saliva or NP sample was directly collected into a tube containing the composition and was applied to the electroactive indicator (65° C.) then the display module was used to monitor the redox current reduction in real-time.

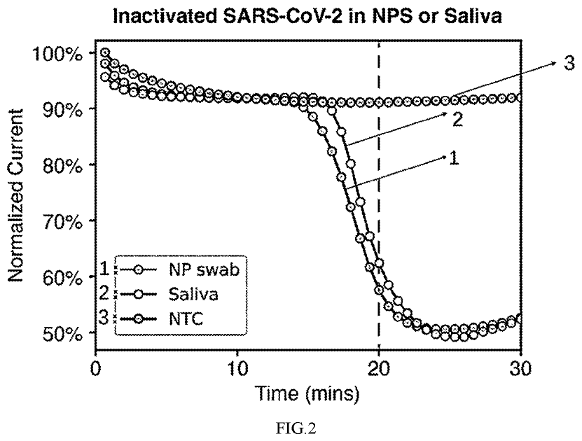

Both the saliva and NP samples showed ˜40% current reduction (80% of the maximum signal reduction observed in buffer) around 20 minutes and ˜50% current reduction (the maximum signal reduction observed in buffer) within 25 minutes (FIG. 2). It was demonstrated the simplicity of the system and selected a readout time of 20 min for subsequent LoD analysis. In FIG. 2, the lines 1-3 are corresponding to the result of the NP swab, saliva and NTC.

The composition comprises the following: DTT (about 0.15 Unit/μl), R8759 (Sigma Aldrich) (about 0.05 μg/μl), the reverse transcriptase (about 0.3 Unit/μl), the isothermal amplification buffer, Tween 20 (about 0.1 (v/v)), each dNTP (about 1.4 mM) and the N4 primer (N4-FIP about 1.6 μM; N4-BIP about 1.6 μM; N4-F3 about 0.2 μM; N4-B3 about 0.2 μM; N4-LF about 0.4 μM; N4-LB about 0.2 μM).

### Example 3

The system of the present application has nearly completed the assay validation step required by FDA.

In the Policy for Coronavirus Disease-2019 Tests, the FDA has articulated the validation studies in four areas required to support an EUA: (1) Limit of Detection, (2) Inclusivity, (3) Cross-reactivity, and (4) Clinical Evaluation.

### 1 Limit of Detection.

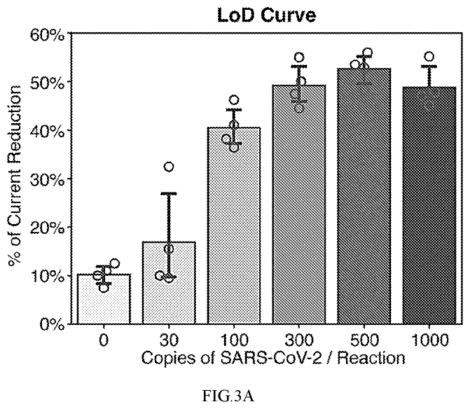

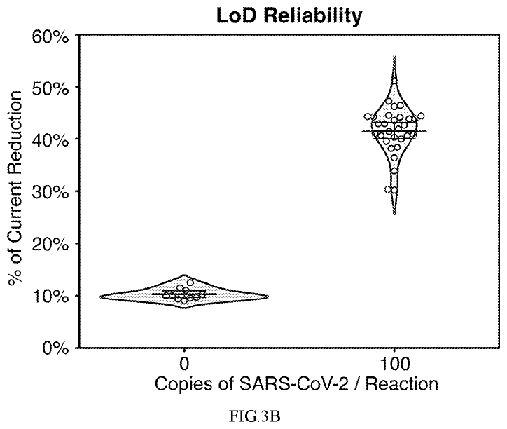

The LOD determination study was conducted using contrived samples. Heat-inactivated SARS-COV-2 (ATCC, VR-1986HK) was spiked into pooled negative saliva human clinical specimens. Saliva as the clinical matrix was chosen because it represents a more challenging matrix compared to swabs and LOD results in saliva will satisfy both saliva and swab matrices in our EUA request. A preliminary LOD was determined by testing serial dilutions of inactivated SARS-COV-2 spiked into pooled negative saliva matrix and tested with four replicates per concentration, and then confirmed with 30 replicates at the LOD concentration. The LOD of the system of assay was determined to be 100 copies of SARS-COV-2 per reaction (FIGS. 3A-3B), at which 30/30 replicates are positive, exceeding FDA's requirement of >19/20 replicates being positive. It was chosen a cut-off to be Current Reduction=20%, which represents 4 standard deviations above the NTC.

In FIGS. 3A-3B, SARS-COV-2 RNA Control (Twist Bioscience, 102024) spiked into saliva was used for input. A) All four replicates could be positively detected down to 100 copies of inactivated SARS-COV-2 per reaction. B) To confirm the determined LOD of 100 copies of inactivated SARS-COV-2 per reaction in saliva sample, the system of the present application yielded positive results for 30 replicates.

### 2. Inclusivity (Analytical Sensitivity).

It was completed in silico analysis of inclusivity to demonstrate the N4 primer set used in the system of the present application is able to detect all SARS-COV-2 sequences. As of June 13th, there are 45,869 full genome data for SARS-COV-2 virus submitted to the GITHE gene database. The dataset was refined by removing duplicate and ambiguous sequencing data (e.g. N's), and the resulting 41,748 complete strains (length >29,000 bp) are used to analyze the inclusivity of ACE. Table 1 summarizes the homology between 41,748 SARS-COV-2 sequences and the N4 primer set used in ACE. The N4 primer set has 100% homology with 98.19% of all strains. Of the 757 mismatches, 3.04% due to sequencing error and most are due to a single mismatch (96.43%) and that are relatively far from extension start. Based on melting temperature analysis, none of these mismatches found are predicted to have a negative impact on the performance of the assay, and the N4 primer set is predicted to detect 100% of SARS-COV-2 sequences currently available for analysis.

TABLE 1
Table1. In silico Inclusivity Analysis.

| | | | 100% | 100% homology for individual primer fragments (%) | | | | | | | |
|---|---|---|---|---|---|---|---|---|---|---|---|
| Primer | Seqs | Mismatch | Homology | F1c | F2 | F3 | B1c | B2 | B3 | LB | LF |
| N4 | 41748 | 757 | 98.19% | 99.74 | 99.45 | 99.80 | 99.71 | 99.86 | 99.86 | 99.57 | 99.80 |

The N4 primer comprises the following sequences: SEQ ID NO. 3 (FIP); SEQ ID NO. 4 (BIP); SEQ ID NO.1 (F3); SEQ ID NO. 2 (B3); SEQ ID NO. 5 (LF); and SEQ ID NO. 6 (LB); and SEQ ID NO. 3 (FIP) is consisted of two fragments SEQ ID NO. 7 (F1C) and SEQ ID NO.8(F2);

and SEQ ID NO. 4 (BIP) may comprise or be consisted of two fragments SEQ ID NO. 9 (B1C) and SEQ ID NO. 10 (B2).

### 3. Cross-Reactivity (Analytical Specificity).

It was conducted in silico analysis of cross-reactivity to demonstrate that ACE assay does not react with a list of related pathogens, common disease agents and flora that are likely to be encountered in the clinical specimen, recommended by FDA (Table 2). SARS-COV is the only organism identified as potentially cross-reactive with the N4 primer set where primer homology exceeds 80% (criteria defined by FDA). Although it cannot be completely ruled out the possibility that the detection of the system of present application may cross-react with SARS-COV, the low prevalence of SARS-COV renders the observation of cross-reactivity unlikely. Indeed, SARS-COV has not been detected in the human population since 2004. This standard has been deemed acceptable to FDA for previous EUA (e.g. Abbott's ID NOW, Mesa Biotech's Accula, and Cepheid's Xpert Xpress SARS-COV-2 Tests).

TABLE 2
In Silico Cross-Reactivity

| Organism | Homology |
|---|---|
| H. Coronavirus 229E (NC_002645) | 60% |
| H. Coronavirus 229E (MN369046) | 59% |
| H. Coronavirus OC43 (KX344031.1) | 48% |
| H. Coronavirus OC43 (NC_006213) | 48% |
| H. Coronavirus HKU1 (NC_006577) | 52% |
| H. Coronavirus HKU1 (MK167038) | 58% |
| H. Coronavirus HKU1 (MH940245) | 60% |
| H. Coronavirus NL63 (NC005831) | 59% |
| SARS Coronavirus (NC_004718) | 86% |
| MERS (NC_019843) | 62% |
| Adenovirus (AC_000017) | 60% |
| H. Metapneumovirus (NC_039199) | 62% |
| H. Parainfluenza 1 (AF457102) | 56% |
| H. Parainfluenza 4a (NC_021928) | 60% |
| H. Parainfluenza 4b (MN306032) | 60% |
| Influenza A (NC_026435) | 54% |
| Influenza B (NC_002205) | 60% |
| H. rhinovirus (NC_038311) | 47% |
| Chlamydia pneumoniae | 57% |
| Legionella pneumophila | 66% |
| Mycobacterium tuberculosis | 63% |
| Streptococcus pneumoniae | 68% |
| Streptococcus pyogenes | 64% |
| Bordetella pertussis | 40% |
| Mycoplasma pneumoniae | 57% |
| Pneumocystis jirovecii | 59% |
| Candida albicans | 66% |
| Pseudomonas aeruginosa | 69% |
| Staphylococcus epidermidis | 63% |
| Streptococcus salivarius | 64% |

### Example 4

The current system starts from saliva or nasopharyngeal swab collection into a barcoded sample collection tube containing the inhibitor of RNase A, RNase B and/or RNase C of the present application, the reverse transcriptase of the present application, the dNTP of the present application and the Tween 20 of the present application, allowing direct testing from unprocessed patient samples and making it readily deployable to home and public setting and used by lay-persons.

It was validated by not successfully detecting all 100 copies of heat-inactivated SARS-COV-2 virus (ATCC, VR-1986HK) spiked into pooled healthy saliva or nasopharyngeal (NP) swab samples.

The composition is consisted of: DTT (about 0.15 Unit/μl), the reverse transcriptase (about 0.3 Unit/μl), the isothermal amplification buffer, Tween 20 (about 0.1 (v/v)), each dNTP (about 1.4 mM) and the N4 primer (N4-FIP about 1.6 μM; N4-BIP about 1.6 μM; N4-F3 about 0.2 μM; N4-B3 about 0.2 μM; N4-LF about 0.4 μM; N4-LB about 0.2 μM).

### Example 5

The current system starts from saliva or nasopharyngeal swab collection into a barcoded sample collection tube containing tRNA of the present application, the reverse transcriptase of the present application, the dNTP of the present application and the Tween 20 of the present application, allowing direct testing from unprocessed patient samples and making it readily deployable to home and public setting and used by lay-persons.

It was validated by not successfully detecting all 100 copies of heat-inactivated SARS-COV-2 virus (ATCC, VR-1986HK) spiked into pooled healthy saliva or nasopharyngeal (NP) swab samples.

The composition is consisted of: R8759 (Sigma Aldrich) (about 0.05 μg/μl), the reverse transcriptase (about 0.3 Unit/μl), the isothermal amplification buffer, Tween 20 (about 0.1 (v/v)), each dNTP (about 1.4 mM) and the N4 primer (N4-FIP about 1.6 μM; N4-BIP about 1.6 μM; N4-F3 about 0.2 μM; N4-B3 about 0.2 μM; N4-LF about 0.4 μM; N4-LB about 0.2 μM).

### Example 6

24 sets of LAMP primers targeting different regions of the SARS-COV-2 genome were tested. After thorough testing, a novel LAMP primer set for amplifying N gene of the SARS-COV-2, named Aptitude N7 primer set was identified, with which ˜100 copies of the SARS-COV-2 RNA (Twist Bioscience, 102024) per sample can be detected and yield maximum electrochemical current signal reduction (˜50%) within 30 minutes. The result was measured by real-time electrochemical current across a range of concentrations (0 to 100 copies of SARS-COV-2 viral RNA per reaction) of SARS-CoV-2 genomic standards.

Moreover, the N7 primer set showed no non-specific amplification and negligible signal reduction for the no template control (NTC), showing superior specificity than the established LAMP primer set 46 and another set of in-house primer (N5) for the N gene.

The N7 primer comprises the following sequences: SEQ ID NO. 45 (FIP); SEQ ID NO. 46 (BIP); SEQ ID NO. 43 (F3); SEQ ID NO. 44 (B3); SEQ ID NO. 57 (LF); and SEQ ID NO. 58 (LB).

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A composition for detecting a target nucleic acid in a sample, said composition comprising: a nuclease inhibitor and a decoy oligonucleotide, wherein said decoy oligonucleotide comprises tRNAs, and wherein said nuclease inhibitor comprises an RNase inhibitor.

2-9. (canceled)

10. The composition of claim 1, wherein said RNase inhibitor comprises a protein-based RNase inhibitor and/or a chemical-based RNase inhibitor, and wherein said RNase inhibitor is capable of specifically inhibiting an activity of RNase A, RNase B and/or RNase C.

11-14. (canceled)

15. The composition of claim 1, wherein said nuclease inhibitor comprises one or more of the following: oligovinylsulfonic acid (OVA), aurintricarboxylic acid (ATA), aflatoxin, 2-nitro-5-thiocyanobenzoic acid, iodoacetate, N-bromosuccinimide, pchloromercuribenzoate, diethyl pyrocarbonate, ethanol, formamide, guanidinium thiocyanate (GdnSCN), dinitrofluorobenzene, decanavanate, polyvinylsufonic acid, hydrobenzoinphosphate, phenylphosphate, putrescine, haloacetate, dinitrofluorobenzene, phenylglyoxal, bromopyruvic, hydroxylamine-oxygen-cupric ion, a vanadyl complex, 8-amino-5-(4′-hydroxybiphenyl-4-ylazo)-naphthalene-2-sulfonate, 6-hydroxy-5-(2-hydroxy-3,5-dinitro-phenylazo)-naphthalene-2-sulfonate, 3,3′-dimethylbiphenyl-4,4′-bis(2-amino-naphthylazo-6-sulfonate), 4,4′-dicarboxy-3,3′-bis(naphthylamido)-diphenylmethanone, 3,3′-dicarboxy-4,4′-bis(4-biphenylamido) diphenylmethane, 3,3′-dicarboxy-4,4′-bis(3-nitrophenylamido)diphenylmethane, RNase Inhibitor-Murine (NEB M0314), Protector RNase Inhibitor (Roche, RNAINH-RO)β-mercaptoethanol, RNAsecure (Thermo Fisher Scientific, AM7005), dithiothreitol (DTT), dithioerythritol (DTE), Tris (2-carboxyethyl) phosphine (TCEP) glutathione and cysteine.

16. The composition of claim 1, wherein said nuclease inhibitor is present in a concentration of about 0.01 Unit/μl to about 1 Unit/μl.

17-18. (canceled)

19. The composition of claim 1, wherein said decoy oligonucleotide is present in a concentration of about 0.01 μg/μl to about 1 μg/μl.

20-21. (canceled)

22. The composition of claim 1, further comprising an agent capable of amplifying a nucleic acid, wherein said agent capable of amplifying a nucleic acid comprises a reverse transcriptase.

23-24. (canceled)

25. The composition of claim 22, wherein said agent capable of amplifying a nucleic acid is present in a concentration of about 0.1 Unit/μl to about 5 Unit/μl.

26-30. (canceled)

31. The composition of claim 1, further comprising a buffer, wherein said buffer comprises an isothermal amplification buffer.

32. (canceled)

33. The composition of claim 1, further comprising a lysing agent, wherein said lysing agent comprises a surfactant.

34-37. (canceled)

38. The composition of claim 33, wherein said lysing agent is present in a concentration of about 0.01% (v/v) to about 1.5% (v/v).

39-40. (canceled)

41. The composition of claim 1, further comprising dNTPs, wherein each dNTP is present in a concentration of about 0.5 mM to about 5 mM.

42- 43. (canceled)

44. The composition of claim 1, further comprising one or more primers specific for said target nucleic acid, wherein said one or more primers are suitable for amplifying said target nucleic acid in an isothermal amplification, and wherein said isothermal amplification comprises NASBA (nucleic acid sequence-based amplification), loop-mediated isothermal amplification (LAMP), recombinase polymerase amplification (RPA), and/or helicase-dependent amplification (HDA).

45-48. (canceled)

49. The composition of claim 44, wherein said primer contains at most one fragment of 4 consecutive identical single nucleotides, and wherein said primer does not form any secondary structure with a ΔG lower than about −4 kcal/mol, and/or said primer does not comprise a 3′ end region having 6 or more nucleotides that are reverse complementary to a sequence within the same primer or within another primer of said one or more primers.

50-58. (canceled)

59. The composition of claim 44, wherein said target nucleic acid is a SARS-COV-2-associated nucleic acid, and said one or more primers comprise primers 1-6, wherein said primer 1 comprises a nucleotide sequence as set forth in any of SEQ ID NO.3, SEQ ID NO.13, SEQ ID NO.23, SEQ ID NO.33, SEQ ID NO.45, SEQ ID NO.63, SEQ ID NO.73, SEQ ID NO.83, SEQ ID NO.93, SEQ ID NO.106, SEQ ID NO.116, SEQ ID NO.126, SEQ ID NO.136, SEQ ID NO.146, wherein said primer 2 comprises a nucleotide sequence as set forth in any of SEQ ID NO.4, SEQ ID NO.14, SEQ ID NO.34, SEQ ID NO.46, SEQ ID NO.64, SEQ ID NO.74, SEQ ID NO.84, SEQ ID NO.94, SEQ ID NO.107, SEQ ID NO.117, SEQ ID NO.127, SEQ ID NO.137, SEQ ID NO.147 and SEQ ID NO.157, wherein said primer 3 comprises a nucleotide sequence as set forth in any of SEQ ID NO.1, SEQ ID NO.11, SEQ ID NO.31, SEQ ID NO.41, SEQ ID NO.43, SEQ ID NO.55, SEQ ID NO. 61, SEQ ID NO. 71, SEQ ID NO. 81, SEQ ID NO.91, SEQ ID NO.101, SEQ ID NO.104, SEQ ID NO.114, SEQ ID NO.124, SEQ ID NO.134, SEQ ID NO.144, SEQ ID NO.154 AND SEQ I.154 and SEQ ID NO.163, wherein said primer 4 comprises a nucleotide sequence as set forth in any of SEQ ID NO.2, SEQ ID NO.12, SEQ ID NO.32, SEQ ID NO.42, SEQ ID NO.44, SEQ ID NO.56, SEQ ID NO.62, SEQ ID NO.72, SEQ ID NO.82, SEQ ID NO.92, SEQ ID NO.102, SEQ ID NO.105, SEQ ID NO.115, SEQ ID NO.125, SEQ ID NO.135, SEQ ID NO.145 and SEQ ID NO.155.155, wherein said primer 5 comprises a nucleotide sequence as set forth in any of SEQ ID NO.5, SEQ ID NO.15, SEQ ID NO.35, SEQ ID NO.47, SEQ ID NO.53, SEQ ID NO.55, SEQ ID NO.57, SEQ ID NO.59, SEQ ID NO.65, SEQ ID NO.75, SEQ ID NO.85, SEQ ID NO.95, SEQ ID NO.103, SEQ ID NO.108, SEQ ID NO.118, SEQ ID NO.128, SEQ ID NO.138, SEQ ID NO.148 SEQ ID NO.148 and SEQ ID NO.158, and wherein said primer 6 comprises a nucleotide sequence as set forth in any of SEQ ID NO.6, SEQ ID NO.16, SEQ ID NO.36, SEQ ID NO.48, SEQ ID NO.54, SEQ ID NO.56, SEQ ID NO.58, SEQ ID NO.60, SEQ ID NO.66, SEQ ID NO.76, SEQ ID NO.86, SEQ ID NO.96, SEQ ID NO. 109, SEQ ID NO.119, SEQ ID NO. 129, SEQ ID NO.139 and SEQ ID NO.149.

60. (canceled)

61. The composition of claim 59, wherein said primer 1 is present in a concentration of about luM to about 2 μM, wherein said primer 2 is present in a concentration of about luM to about 2 μM, wherein said primer 3 is present in a concentration of about 0.1 μM to about 0.5 μM, wherein said primer 4 is present in a concentration of about 0.1 μM to about 0.5 μM, wherein said primer 5 is present in a concentration of about 0.3 μM to about 0.8 μM, and wherein said primer 6 is present in a concentration of about 0.3 μM to about 0.8 μM.

62. The composition of claim 1, wherein said sample is a biological sample, wherein said sample is selected from the group consisting of: nasopharyngeal swab, blood, serum, urine, saliva, tissue, cell, organ, a fraction or portion thereof, and any combination of the foregoing, and wherein said sample is a crude sample derived directly from a subject without being processed.

63-64. (canceled)

65. The composition of any one of claims | 64claim 1, wherein said target nucleic acid in said sample has not been subjected to amplification and/or purification.

66-67. (canceled)

68. An isolated oligonucleotide, comprising a sequence as set forth in any of SEQ ID Nos. 1-20, 31-183.

69-83. (canceled)

84. A primer set, comprising primers 1-6, wherein said primer 1 comprises a nucleotide sequence as set forth in any of SEQ ID NO.3, SEQ ID NO.13, SEQ ID NO.33, SEQ ID NO.45, SEQ ID NO.63, SEQ ID NO.73, SEQ ID NO.83, SEQ ID NO.93, SEQ ID NO.106, SEQ ID NO.116, SEQ ID NO.126, SEQ ID NO.136, SEQ ID NO.146 and SEQ ID NO.156, wherein said primer 2 comprises a nucleotide sequence as set forth in any of SEQ ID NO.4, SEQ ID NO.14, SEQ ID NO.34, SEQ ID NO.46, SEQ ID NO.64, SEQ ID NO.74, SEQ ID NO.84, SEQ ID NO.94, SEQ ID NO.107, SEQ ID NO.117, SEQ ID NO.127, SEQ ID NO.137, SEQ ID NO.147 and SEQ ID NO.157, wherein said primer 3 comprises a nucleotide sequence as set forth in any of SEQ ID NO.1, SEQ ID NO.11, SEQ ID NO.31, SEQ ID NO.41, SEQ ID NO.43, SEQ ID NO.55, SEQ ID NO. 61, SEQ ID NO. 71, SEQ ID NO. 81, SEQ ID NO.91, SEQ ID NO.101, SEQ ID NO.104, SEQ ID NO.114, SEQ ID NO.124, SEQ ID NO.134, SEQ ID NO.144, SEQ ID NO.154 AND SEQ I.154 and SEQ ID NO.163, wherein said primer 4 comprises a nucleotide sequence as set forth in any of SEQ ID NO.2, SEQ ID NO.12, SEQ ID NO.32, SEQ ID NO.42, SEQ ID NO.44, SEQ ID NO.56, SEQ ID NO.62, SEQ ID NO.72, SEQ ID NO.82, SEQ ID NO.92, SEQ ID NO.102, SEQ ID NO. 105, SEQ ID NO.115, SEQ ID NO.125, SEQ ID NO. 135, SEQ ID NO. 145 and SEQ ID NO.155.155, wherein said primer 5 comprises a nucleotide sequence as set forth in any of SEQ ID NO.5, SEQ ID NO.15, SEQ ID NO.35, SEQ ID NO.47, SEQ ID NO.53, SEQ ID NO.55, SEQ ID NO.57, SEQ ID NO.59, SEQ ID NO.65, SEQ ID NO.75, SEQ ID NO.85, SEQ ID NO.95, SEQ ID NO.103, SEQ ID NO.108, SEQ ID NO.118, SEQ ID NO.128, SEQ ID NO.138, SEQ ID NO.148 SEQ ID NO.148 and SEQ ID NO.158, and wherein said primer 6 comprises a nucleotide sequence as set forth in any of SEQ ID NO.6, SEQ ID NO.16, SEQ ID NO.36, SEQ ID NO.48, SEQ ID NO.54, SEQ ID NO.56, SEQ ID NO.58, SEQ ID NO.60, SEQ ID NO.66, SEQ ID NO.76, SEQ ID NO.86, SEQ ID NO.96, SEQ ID NO.109, SEQ ID NO.119, SEQ ID NO.129, SEQ ID NO. 139 and SEQ ID NO.149.

85-91. (canceled)

92. A method for detecting a target nucleic acid in a sample, comprising administering the composition of claim 1.

93-102. (canceled)


SEQUENCE LISTING (183 sequences)

SEQ ID NO: 1 - DNA, 19 bp, Artificial Sequence
  N4-F3
gccaaaaggcttctacgca

SEQ ID NO: 2 - DNA, 20 bp, Artificial Sequence
  N4-B3
tttgctctcaagctggttca

SEQ ID NO: 3 - DNA, 40 bp, Artificial Sequence
  N4-FIP; N11-FIP
tcccctactgctgcctggagcagtcaagcctcttctcgtt

SEQ ID NO: 4 - DNA, 42 bp, Artificial Sequence
  N4-BIP; N11-BIP
tctcctgctagaatggctggcatctgtcaagcagcagcaaag

SEQ ID NO: 5 - DNA, 24 bp, Artificial Sequence
  N4-LF
cttgaactgttgcgactacgtgat

SEQ ID NO: 6 - DNA, 19 bp, Artificial Sequence
  N4-LB; N11-LB
atggcggtgatgctgctct

SEQ ID NO: 7 - DNA, 20 bp, Artificial Sequence
  N4-F1c; N11-F1c
tcccctactgctgcctggag

SEQ ID NO: 8 - DNA, 20 bp, Artificial Sequence
  N4-F2; N11-F2
cagtcaagcctcttctcgtt

SEQ ID NO: 9 - DNA, 22 bp, Artificial Sequence
  N4-B1c; N11-B1c
tctcctgctagaatggctggca

SEQ ID NO: 10 - DNA, 20 bp, Artificial Sequence
  N4-B2; N11-B2
tctgtcaagcagcagcaaag

SEQ ID NO: 11 - DNA, 22 bp, Artificial Sequence
  N5-F3
cttcctcaaggaacaacattgc

SEQ ID NO: 12 - DNA, 19 bp, Artificial Sequence
  N5-B3
atctgtcaagcagcagcaa

SEQ ID NO: 13 - DNA, 44 bp, Artificial Sequence
  N5-FIP
gcgactacgtgatgaggaacgagaaggcttctacgcagaaggga

SEQ ID NO: 14 - DNA, 38 bp, Artificial Sequence
  N5-BIP
actccaggcagcagtagggggagcagcatcaccgccat

SEQ ID NO: 15 - DNA, 17 bp, Artificial Sequence
  N5-LF
aggcttgactgccgcct

SEQ ID NO: 16 - DNA, 20 bp, Artificial Sequence
  N5-LB
tcctgctagaatggctggca

SEQ ID NO: 17 - DNA, 24 bp, Artificial Sequence
  N5-F1c
gcgactacgtgatgaggaacgaga

SEQ ID NO: 18 - DNA, 20 bp, Artificial Sequence
  N5-F2
aggcttctacgcagaaggga

SEQ ID NO: 19 - DNA, 20 bp, Artificial Sequence
  N5-B1c
actccaggcagcagtagggg

SEQ ID NO: 20 - DNA, 18 bp, Artificial Sequence
  N5-B2
gagcagcatcaccgccat

SEQ ID NO: 21 - DNA, 19 bp, Artificial Sequence
  NA-F3
tggctactaccgaagagct

SEQ ID NO: 22 - DNA, 20 bp, Artificial Sequence
  NA-B3
tgcagcattgttagcaggat

SEQ ID NO: 23 - DNA, 41 bp, Artificial Sequence
  NA-FIP
tctggcccagttcctaggtagtccagacgaattcgtggtgg

SEQ ID NO: 24 - DNA, 40 bp, Artificial Sequence
  NA-BIP
agacggcatcatatgggttgcacgggtgccaatgtgatct

SEQ ID NO: 25 - DNA, 25 bp, Artificial Sequence
  NA-LF
ggactgagatctttcattttaccgt

SEQ ID NO: 26 - DNA, 21 bp, Artificial Sequence
  NA-LB
actgagggagccttgaataca

SEQ ID NO: 27 - DNA, 22 bp, Artificial Sequence
  NA-F1c
tctggcccagttcctaggtagt

SEQ ID NO: 28 - DNA, 19 bp, Artificial Sequence
  NA-F2
ccagacgaattcgtggtgg

SEQ ID NO: 29 - DNA, 22 bp, Artificial Sequence
  NA-B1c
agacggcatcatatgggttgca

SEQ ID NO: 30 - DNA, 18 bp, Artificial Sequence
  NA-B2
cgggtgccaatgtgatct

SEQ ID NO: 31 - DNA, 19 bp, Artificial Sequence
  M1-F3
tgctatcgcaatggcttgt

SEQ ID NO: 32 - DNA, 22 bp, Artificial Sequence
  M1-B3
agtgatttctttaggcaggtcc

SEQ ID NO: 33 - DNA, 46 bp, Artificial Sequence
  M1-FIP
ggtctggtcagaatagtgccatgggttccatgtggtcattcaatcc

SEQ ID NO: 34 - DNA, 43 bp, Artificial Sequence
  M1-BIP
agtgaactcgtaatcggagctgtgagcgtcctagatggtgtcc

SEQ ID NO: 35 - DNA, 25 bp, Artificial Sequence
  M1-LF
agtggcacgttgagaagaatgttag

SEQ ID NO: 36 - DNA, 24 bp, Artificial Sequence
  M1-LB
ccttcgtggacatcttcgtattgc

SEQ ID NO: 37 - DNA, 24 bp, Artificial Sequence
  M1-F1c
ggtctggtcagaatagtgccatgg

SEQ ID NO: 38 - DNA, 22 bp, Artificial Sequence
  M1-F2
gttccatgtggtcattcaatcc

SEQ ID NO: 39 - DNA, 24 bp, Artificial Sequence
  M1-B1c
agtgaactcgtaatcggagctgtg

SEQ ID NO: 40 - DNA, 19 bp, Artificial Sequence
  M1-B2
agcgtcctagatggtgtcc

SEQ ID NO: 41 - DNA, 19 bp, Artificial Sequence
  M2-F3
ttcagactgtttgcgcgta

SEQ ID NO: 42 - DNA, 21 bp, Artificial Sequence
  M2-B3
ctttaggcaggtccttgatgt

SEQ ID NO: 43 - DNA, 19 bp, Artificial Sequence
  N7-F3
ccgcattacgtttggtgga

SEQ ID NO: 44 - DNA, 22 bp, Artificial Sequence
  N7-B3
catctggactgctattggtgtt

SEQ ID NO: 45 - DNA, 41 bp, Artificial Sequence
  N7-FIP
ccttggggccgacgttgtttcagattcaactggcagtaacc

SEQ ID NO: 46 - DNA, 40 bp, Artificial Sequence
  N7-BIP
tactgcgtcttggttcaccgcgccttgtcctcgagggaat

SEQ ID NO: 47 - DNA, 19 bp, Artificial Sequence
  N7-LF
gccccactgcgttctccat

SEQ ID NO: 48 - DNA, 24 bp, Artificial Sequence
  N7-LB
tctcactcaacatggcaaggaaga

SEQ ID NO: 49 - DNA, 19 bp, Artificial Sequence
  N7-F1c
ccttggggccgacgttgtt

SEQ ID NO: 50 - DNA, 22 bp, Artificial Sequence
  N7-F2
tcagattcaactggcagtaacc

SEQ ID NO: 51 - DNA, 21 bp, Artificial Sequence
  N7-B1c
tactgcgtcttggttcaccgc

SEQ ID NO: 52 - DNA, 19 bp, Artificial Sequence
  N7-B2
gccttgtcctcgagggaat

SEQ ID NO: 53 - DNA, 20 bp, Artificial Sequence
  N7-LF2
cgccccactgcgttctccat

SEQ ID NO: 54 - DNA, 25 bp, Artificial Sequence
  N7-LB2
tctcactcaacatggcaaggaagac

SEQ ID NO: 55 - DNA, 21 bp, Artificial Sequence
  N7-LF3
gcgccccactgcgttctccat

SEQ ID NO: 56 - DNA, 26 bp, Artificial Sequence
  N7-LB3
tctcactcaacatggcaaggaagacc

SEQ ID NO: 57 - DNA, 22 bp, Artificial Sequence
  N7-LF4
cgcgccccactgcgttctccat

SEQ ID NO: 58 - DNA, 27 bp, Artificial Sequence
  N7-LB4
tctcactcaacatggcaaggaagacct

SEQ ID NO: 59 - DNA, 24 bp, Artificial Sequence
  N7-LF5
atcgcgccccactgcgttctccat

SEQ ID NO: 60 - DNA, 29 bp, Artificial Sequence
  N7-LB5
tctcactcaacatggcaaggaagacctta

SEQ ID NO: 61 - DNA, 22 bp, Artificial Sequence
  S1-F3
atcaaaaccaagcaagaggtca

SEQ ID NO: 62 - DNA, 21 bp, Artificial Sequence
  S1-B3
aggtccaaccagaagtgattg

SEQ ID NO: 63 - DNA, 44 bp, Artificial Sequence
  S1-FIP
ttgtgcacaaatgaggtctctagcagacacttgcagatgctggc

SEQ ID NO: 64 - DNA, 45 bp, Artificial Sequence
  S1-BIP
taacggccttactgttttgccacccgctaacagtgcagaagtgta

SEQ ID NO: 65 - DNA, 25 bp, Artificial Sequence
  S1-LF
gcaatatcaccaaggcaatcaccat

SEQ ID NO: 66 - DNA, 25 bp, Artificial Sequence
  S1-LB
tgctcacagatgaaatgattgctca

SEQ ID NO: 67 - DNA, 25 bp, Artificial Sequence
  S1-F1c
ttgtgcacaaatgaggtctctagca

SEQ ID NO: 68 - DNA, 19 bp, Artificial Sequence
  S1-F2
gacacttgcagatgctggc

SEQ ID NO: 69 - DNA, 24 bp, Artificial Sequence
  S1-B1c
taacggccttactgttttgccacc

SEQ ID NO: 70 - DNA, 21 bp, Artificial Sequence
  S1-B2
cgctaacagtgcagaagtgta

SEQ ID NO: 71 - DNA, 19 bp, Artificial Sequence
  N9-F3
atcaaaacaacgtcggccc

SEQ ID NO: 72 - DNA, 21 bp, Artificial Sequence
  N9-B3
gcccagttcctaggtagtaga

SEQ ID NO: 73 - DNA, 41 bp, Artificial Sequence
  N9-FIP
acgccttgtcctcgagggaattgtcttggttcaccgctctc

SEQ ID NO: 74 - DNA, 46 bp, Artificial Sequence
  N9-BIP
tagcagtccagatgaccaaattggctcattttaccgtcaccaccac

SEQ ID NO: 75 - DNA, 23 bp, Artificial Sequence
  N9-LF
aggtcttccttgccatgttgagt

SEQ ID NO: 76 - DNA, 24 bp, Artificial Sequence
  N9-LB; N14-LB
tactaccgaagagctaccagacga

SEQ ID NO: 77 - DNA, 22 bp, Artificial Sequence
  N9-F1c
acgccttgtcctcgagggaatt

SEQ ID NO: 78 - DNA, 19 bp, Artificial Sequence
  N9-F2
gtcttggttcaccgctctc

SEQ ID NO: 79 - DNA, 25 bp, Artificial Sequence
  N9-B1c
tagcagtccagatgaccaaattggc

SEQ ID NO: 80 - DNA, 21 bp, Artificial Sequence
  N9-B2
tcattttaccgtcaccaccac

SEQ ID NO: 81 - DNA, 22 bp, Artificial Sequence
  N10-F3
ggtggtgacggtaaaatgaaag

SEQ ID NO: 82 - DNA, 21 bp, Artificial Sequence
  N10-B3
cgtagaagccttttggcaatg

SEQ ID NO: 83 - DNA, 46 bp, Artificial Sequence
  N10-FIP
cctcagttgcaacccatatgatgcctactacctaggaactgggcca

SEQ ID NO: 84 - DNA, 46 bp, Artificial Sequence
  N10-BIP
acaccaaaagatcacattggcaccccttgaggaagttgtagcacga

SEQ ID NO: 85 - DNA, 22 bp, Artificial Sequence
  N10-LF
gcaccatagggaagtccagctt

SEQ ID NO: 86 - DNA, 24 bp, Artificial Sequence
  N10-LB
gcaatcctgctaacaatgctgcaa

SEQ ID NO: 87 - DNA, 25 bp, Artificial Sequence
  N10-F1c
cctcagttgcaacccatatgatgcc

SEQ ID NO: 88 - DNA, 21 bp, Artificial Sequence
  N10-F2
tactacctaggaactgggcca

SEQ ID NO: 89 - DNA, 25 bp, Artificial Sequence
  N10-B1c
acaccaaaagatcacattggcaccc

SEQ ID NO: 90 - DNA, 21 bp, Artificial Sequence
  N10-B2
cttgaggaagttgtagcacga

SEQ ID NO: 91 - DNA, 19 bp, Artificial Sequence
  O6-F3
tgctaatgaccctgtgggt

SEQ ID NO: 92 - DNA, 22 bp, Artificial Sequence
  O6-B3
ttgtagatgtcaaaagccctgt

SEQ ID NO: 93 - DNA, 43 bp, Artificial Sequence
  O6-FIP
catcagctgactgaagcatgggtgtctgcggtatgtggaaagg

SEQ ID NO: 94 - DNA, 43 bp, Artificial Sequence
  O6-BIP
aacgggtttgcggtgtaagtgccatcagtactagtgcctgtgc

SEQ ID NO: 95 - DNA, 24 bp, Artificial Sequence
  O6-LF
cggagttgatcacaactacagcca

SEQ ID NO: 96 - DNA, 19 bp, Artificial Sequence
  O6-LB
agcccgtcttacaccgtgc

SEQ ID NO: 97 - DNA, 23 bp, Artificial Sequence
  O6-F1c
catcagctgactgaagcatgggt

SEQ ID NO: 98 - DNA, 20 bp, Artificial Sequence
  O6-F2
gtctgcggtatgtggaaagg

SEQ ID NO: 99 - DNA, 22 bp, Artificial Sequence
  O6-B1c
aacgggtttgcggtgtaagtgc

SEQ ID NO: 100 - DNA, 21 bp, Artificial Sequence
  O6-B2
catcagtactagtgcctgtgc

SEQ ID NO: 101 - DNA, 19 bp, Artificial Sequence
  N11-F3
tgccaaaaggcttctacgc

SEQ ID NO: 102 - DNA, 20 bp, Artificial Sequence
  N11-B3
ttgctctcaagctggttcaa

SEQ ID NO: 103 - DNA, 24 bp, Artificial Sequence
  N11-LF
gaactgttgcgactacgtgatgag

SEQ ID NO: 104 - DNA, 20 bp, Artificial Sequence
  N12-F3
tgtctggtaaaggccaacaa

SEQ ID NO: 105 - DNA, 19 bp, Artificial Sequence
  N12-B3
gtgcaatttgcggccaatg

SEQ ID NO: 106 - DNA, 42 bp, Artificial Sequence
  N12-FIP
gtggcagtacgtttttgccgagaacaaggccaaactgtcact

SEQ ID NO: 107 - DNA, 42 bp, Artificial Sequence
  N12-BIP
aacacaagctttcggcagacgttgattagttcctggtcccca

SEQ ID NO: 108 - DNA, 24 bp, Artificial Sequence
  N12-LF
tcttagaagcctcagcagcagatt

SEQ ID NO: 109 - DNA, 24 bp, Artificial Sequence
  N12-LB
ggtccagaacaaacccaaggaaat

SEQ ID NO: 110 - DNA, 22 bp, Artificial Sequence
  N12-F1c
gtggcagtacgtttttgccgag

SEQ ID NO: 111 - DNA, 20 bp, Artificial Sequence
  N12-F2
aacaaggccaaactgtcact

SEQ ID NO: 112 - DNA, 22 bp, Artificial Sequence
  N12-B1c
aacacaagctttcggcagacgt

SEQ ID NO: 113 - DNA, 20 bp, Artificial Sequence
  N12-B2
tgattagttcctggtcccca

SEQ ID NO: 114 - DNA, 20 bp, Artificial Sequence
  N13-F3
cccaaaatcagcgaaatgca

SEQ ID NO: 115 - DNA, 20 bp, Artificial Sequence
  N13-B3
agccaatttggtcatctgga

SEQ ID NO: 116 - DNA, 40 bp, Artificial Sequence
  N13-FIP
cgttgttttgatcgcgccccattacgtttggtggaccctc

SEQ ID NO: 117 - DNA, 41 bp, Artificial Sequence
  N13-BIP
gcgtcttggttcaccgctctcaattggaacgccttgtcctc

SEQ ID NO: 118 - DNA, 24 bp, Artificial Sequence
  N13-LF
cgttctccattctggttactgcca

SEQ ID NO: 119 - DNA, 25 bp, Artificial Sequence
  N13-LB
ggcaaggaagaccttaaattccctc

SEQ ID NO: 120 - DNA, 20 bp, Artificial Sequence
  N13-F1c
cgttgttttgatcgcgcccc

SEQ ID NO: 121 - DNA, 20 bp, Artificial Sequence
  N13-F2
attacgtttggtggaccctc

SEQ ID NO: 122 - DNA, 21 bp, Artificial Sequence
  N13-B1c
gcgtcttggttcaccgctctc

SEQ ID NO: 123 - DNA, 20 bp, Artificial Sequence
  N13-B2
aattggaacgccttgtcctc

SEQ ID NO: 124 - DNA, 19 bp, Artificial Sequence
  M3-F3
tcgcaatggcttgtcttgt

SEQ ID NO: 125 - DNA, 20 bp, Artificial Sequence
  M3-B3
ggtgtccagcaatacgaaga

SEQ ID NO: 126 - DNA, 40 bp, Artificial Sequence
  M3-FIP
atgaccacatggaacgcgtacgggcttgatgtggctcagc

SEQ ID NO: 127 - DNA, 40 bp, Artificial Sequence
  M3-BIP
caacgtgccactccatggcagaaggatcacagctccgatt

SEQ ID NO: 128 - DNA, 25 bp, Artificial Sequence
  M3-LF
cgcaaacagtctgaaagaagcaatg

SEQ ID NO: 129 - DNA, 24 bp, Artificial Sequence
  M3-LB
gaccagaccgcttctagaaagtga

SEQ ID NO: 130 - DNA, 22 bp, Artificial Sequence
  M3-F1c
atgaccacatggaacgcgtacg

SEQ ID NO: 131 - DNA, 18 bp, Artificial Sequence
  M3-F2
ggcttgatgtggctcagc

SEQ ID NO: 132 - DNA, 20 bp, Artificial Sequence
  M3-B1c
caacgtgccactccatggca

SEQ ID NO: 133 - DNA, 20 bp, Artificial Sequence
  M3-B2
gaaggatcacagctccgatt

SEQ ID NO: 134 - DNA, 22 bp, Artificial Sequence
  M5-F3
gaccgcttctagaaagtgaact

SEQ ID NO: 135 - DNA, 21 bp, Artificial Sequence
  M5-B3
aatcctgtagcgactgtatgc

SEQ ID NO: 136 - DNA, 44 bp, Artificial Sequence
  M5-FIP
ccttgatgtcacagcgtcctagatgcggagctgtgatccttcgt

SEQ ID NO: 137 - DNA, 44 bp, Artificial Sequence
  M5-BIP
atcactgttgctacatcacgaacgcacctgagtcacctgctaca

SEQ ID NO: 138 - DNA, 24 bp, Artificial Sequence
  M5-LF
gtgtccagcaatacgaagatgtcc

SEQ ID NO: 139 - DNA, 21 bp, Artificial Sequence
  M5-LB
caaattgggagcttcgcagcg

SEQ ID NO: 140 - DNA, 25 bp, Artificial Sequence
  M5-F1c
ccttgatgtcacagcgtcctagatg

SEQ ID NO: 141 - DNA, 19 bp, Artificial Sequence
  M5-F2
cggagctgtgatccttcgt

SEQ ID NO: 142 - DNA, 25 bp, Artificial Sequence
  M5-B1c
atcactgttgctacatcacgaacgc

SEQ ID NO: 143 - DNA, 19 bp, Artificial Sequence
  M5-B2
acctgagtcacctgctaca

SEQ ID NO: 144 - DNA, 19 bp, Artificial Sequence
  M4-F3
agctgtgatccttcgtgga

SEQ ID NO: 145 - DNA, 21 bp, Artificial Sequence
  M4-B3
tgctactggaatggtctgtgt

SEQ ID NO: 146 - DNA, 43 bp, Artificial Sequence
  M4-FIP
gcgttcgtgatgtagcaacagtgtcgtattgctggacaccatc

SEQ ID NO: 147 - DNA, 39 bp, Artificial Sequence
  M4-BIP
tgggagcttcgcagcgtgtagccaatcctgtagcgactg

SEQ ID NO: 148 - DNA, 22 bp, Artificial Sequence
  M4-LF
tccttgatgtcacagcgtccta

SEQ ID NO: 149 - DNA, 21 bp, Artificial Sequence
  M4-LB
gcaggtgactcaggttttgct

SEQ ID NO: 150 - DNA, 23 bp, Artificial Sequence
  M4-F1c
gcgttcgtgatgtagcaacagtg

SEQ ID NO: 151 - DNA, 20 bp, Artificial Sequence
  M4-F2
tcgtattgctggacaccatc

SEQ ID NO: 152 - DNA, 20 bp, Artificial Sequence
  M4-B1c
tgggagcttcgcagcgtgta

SEQ ID NO: 153 - DNA, 19 bp, Artificial Sequence
  M4-B2
gccaatcctgtagcgactg

SEQ ID NO: 154 - DNA, 18 bp, Artificial Sequence
  N14-F3
tcggccccaaggtttacc

SEQ ID NO: 155 - DNA, 25 bp, Artificial Sequence
  N14-B3
agtagaaataccatcttggactgag

SEQ ID NO: 156 - DNA, 43 bp, Artificial Sequence
  N14-FIP
ggtgttaattggaacgccttgtcctctgcgtcttggttcaccg

SEQ ID NO: 157 - DNA, 45 bp, Artificial Sequence
  N14-BIP
agcagtccagatgaccaaattggcttcattttaccgtcaccacca

SEQ ID NO: 158 - DNA, 24 bp, Artificial Sequence
  N14-LF
cttccttgccatgttgagtgagag

SEQ ID NO: 159 - DNA, 25 bp, Artificial Sequence
  N14-F1c
ggtgttaattggaacgccttgtcct

SEQ ID NO: 160 - DNA, 18 bp, Artificial Sequence
  N14-F2
ctgcgtcttggttcaccg

SEQ ID NO: 161 - DNA, 24 bp, Artificial Sequence
  N14-B1c
agcagtccagatgaccaaattggc

SEQ ID NO: 162 - DNA, 21 bp, Artificial Sequence
  N14-B2
ttcattttaccgtcaccacca

SEQ ID NO: 163 - DNA, 21 bp, Artificial Sequence
  N15-F3
ggcagtaaccagaatggagaa

SEQ ID NO: 164 - DNA, 19 bp, Artificial Sequence
  RP3-F3
gacgtggcaaatctaggct

SEQ ID NO: 165 - DNA, 21 bp, Artificial Sequence
  RP3-B3
agactggggcattcttttcag

SEQ ID NO: 166 - DNA, 39 bp, Artificial Sequence
  RP3-FIP
ctgctcggcagttggtggactgctgtttgggctctctga

SEQ ID NO: 167 - DNA, 44 bp, Artificial Sequence
  RP3-BIP
tctacagtgaagaaacctcggccatcacacttggctttcttgga

SEQ ID NO: 168 - DNA, 17 bp, Artificial Sequence
  RP3-LF
gcagccttggcgtcact

SEQ ID NO: 169 - DNA, 24 bp, Artificial Sequence
  RP3-LB
aggagatgaagattgtcttccagc

SEQ ID NO: 170 - DNA, 20 bp, Artificial Sequence
  RP3-F1c
ctgctcggcagttggtggac

SEQ ID NO: 171 - DNA, 19 bp, Artificial Sequence
  RP3-F2
tgctgtttgggctctctga

SEQ ID NO: 172 - DNA, 24 bp, Artificial Sequence
  RP3-B1c
tctacagtgaagaaacctcggcca

SEQ ID NO: 173 - DNA, 20 bp, Artificial Sequence
  RP3-B2
tcacacttggctttcttgga

SEQ ID NO: 174 - DNA, 18 bp, Artificial Sequence
  RP4-F3
atccgcaacaactcagcc

SEQ ID NO: 175 - DNA, 19 bp, Artificial Sequence
  RP4-B3
tgggccaccaagagacaat

SEQ ID NO: 176 - DNA, 42 bp, Artificial Sequence
  RP4-FIP
aagagccatatcacggaggggatccacatccgagtcttcagg

SEQ ID NO: 177 - DNA, 39 bp, Artificial Sequence
  RP4-BIP
gacctcggaccagagccatgttacccccaccctcaatgc

SEQ ID NO: 178 - DNA, 25 bp, Artificial Sequence
  RP4-LF
gtcttttcaattacttgggtgtgac

SEQ ID NO: 179 - DNA, 21 bp, Artificial Sequence
  RP4-LB
aagaaaaggcctgttccctgg

SEQ ID NO: 180 - DNA, 22 bp, Artificial Sequence
  RP4-F1c
aagagccatatcacggagggga

SEQ ID NO: 181 - DNA, 20 bp, Artificial Sequence
  RP4-F2
tccacatccgagtcttcagg

SEQ ID NO: 182 - DNA, 21 bp, Artificial Sequence
  RP4-B1c
gacctcggaccagagccatgt

SEQ ID NO: 183 - DNA, 18 bp, Artificial Sequence
  RP4-B2
tacccccaccctcaatgc